SEQUENCE LISTING (32 sequences)

SEQ ID NO: 1 - protein, 108 aa, Artificial Sequence
  Helical bundle 4HBWT
DNIEEVKKMLEKMIKEVKKMLENGEDSEKILKKVKEMIEKILKKVKEDGQDPKMIEEVKKMLEKMIKEVKKMLENGEDSEKILKKVKEMAEKILKKVKELGVGSGWLE

SEQ ID NO: 2 - protein, 108 aa, Artificial Sequence
  Helical bundle 4HB4a
DNIEEVCKMLEKMIKEVKKMLENGEDCEKILKKVKEMIEKILKKVKEDGQDPCMIEEVKKMLEKMIKEVKKMLENGEDCEKILKKVKEMAEKILKKVKELGVGSGWLE

SEQ ID NO: 3 - protein, 108 aa, Artificial Sequence
  Helical bundle 4HB4b
DNIEEVCKMLEKMIKEVKKMLENGEDSEKILKKVCEMIEKILKKVKEDGQDPKMIEEVKKMLEKMIKEVKCMLENGEDSEKILKKVCEMAEKILKKVKELGVGSGWLE

SEQ ID NO: 4 - protein, 108 aa, Artificial Sequence
  Helical bundle 4HB6a
DNIEEVCKMLEKMIKEVKCMLENGEDSEKILKKVCEMIEKILKKVKEDGQDPKMIEEVCKMLEKMIKEVKCMLENGEDSEKILKKVCEMAEKILKKVKELGVGSGWLE

SEQ ID NO: 5 - protein, 108 aa, Artificial Sequence
  Helical bundle 4HB6b
DNIEEVCKMLEKMIKEVKCMLENGEDSECILKKVKEMIECILKKVKEDGQDPKMIEEVKKMLECMIKEVKKMLENGEDSEKILKKVCEMAEKILKKVKELGVGSGWLE

SEQ ID NO: 6 - protein, 108 aa, Artificial Sequence
  Helical bundle 4HB8a
DNIEEVCKMLEKMIKEVCKMLENGEDSECILKKVKEMIECILKKVKEDGQDPKMIEEVCKMLEKMIKEVKCMLENGEDSEKILKCVKEMAEKILKCVKELGVGSGWLE

SEQ ID NO: 7 - protein, 108 aa, Artificial Sequence
  Helical bundle 4HB8b
DNIEEVCKMLEKMICEVKKMLENGEDSEKILCKVKEMIECILKKVKEDGQDPKMIEEVKCMLEKMIKEVKCMLENGEDSECILKKVKEMAECILKKVKELGVGSGWLE

SEQ ID NO: 8 - protein, 108 aa, Artificial Sequence
  Helical bundle 4HB30c
DNIEEVCCELECMICEVCCELENGEDSECILCCVCEEIECILCCVCEDGQDPCMIEEVCCMLECMICEVCCMLENGEDSECILCCVCEMAECILCCVCELGVGSGWLC

SEQ ID NO: 9 - protein, 189 aa, Artificial Sequence
  Helical bundle 4hblongWT
DNEEVKKMLEKMIEEIKKMLEKAIKKVKEMLEKMIKEIKKMLENGEDSEKILKKAKEMAEKILKMVIELAEKILKKAKEMAEKILKKVKELGVDNEEVKKMLEKMIEEIKKMLEKAIKKVKEMLEKMIKEIKKMLENGEDSEKILKKAKEMAEKILKMVIELAEKILKKAKEMAEKILKKVKELGVGGW

SEQ ID NO: 10 - protein, 189 aa, Artificial Sequence
  Helical bundle 4hblong8c
DNECVKKMLEKMIEEIKKMLEKAIKKVKCMLEKMIKEIKKMLENGEDSECILKKAKEMAEKILKMVIELAEKILCKAKEMAEKILKKVKELGVDNEEVKCMLEKMIEEIKKMLEKAIKKVKCMLEKMIKEIKKMLENGEDSEKILCKAKEMAEKILKMVIELAEKILCKAKEMAEKILKKVKELGVGGW

SEQ ID NO: 11 - protein, 189 aa, Artificial Sequence
  Helical bundle 4hblong8c
DNECVKKMLEKMIEEIKKMLECAIKKVKEMLEKMIKEIKCMLENGEDSEKILKKAKEMAECILKMVIELAEKILKKAKCMAEKILKKVKELGVDNECVKKMLEKMIEEIKKMLECAIKKVKEMLEKMIKEIKCMLENGEDSEKILKKACEMAEKILKMVIELAEKILKKACEMAEKILKKVKELGVGGW

SEQ ID NO: 12 - protein, 108 aa, Artificial Sequence
  Helical bundle 4HB6a3ME
DNIEEVCKELEKMIKEVKCELENGEDSEKILKKVCEEIEKILKKVKEDGQDPKMIEEVCKMLEKMIKEVKCMLENGEDSEKILKKVCEMAEKILKKVKELGVGSGWLE

SEQ ID NO: 13 - protein, 101 aa, Artificial Sequence
  Helical bundle HB1P68WT
YGKLNDLLEDLQEVLKNLHKNWHGGKDNLHDVDNHLQNVIEDIHDFMQGGGSGGKLQEMMKEFQQVLDELNNHLQGGKHTVHHIEQNIKEIFHHLEELVHR

SEQ ID NO: 14 - protein, 101 aa, Artificial Sequence
  Helical bundle HB1P686c
YGKLNCLLEDLQEVLCNLHKNWHGGKDNLHDVDNHLQCVIEDIHDFMQGGGSGGCLQEMMKEFQQVLDCLNNHLQGGKHTVHHIEQNICEIFHHLEELVHR

SEQ ID NO: 15 - protein, 232 aa, Artificial Sequence
  Helical bundle HB5CWMWT
DPEDELKRVEKLVKEAEELLRQAKEKGSEEDLEKALRTAEEAAREAKKVLEQAEKEGDPEVALRAVELVVRVAELLLRIAKESGSEEALERALRVAEEAARLAKRVLELAEKQGDPEVALRAVELVVRVAELLLRIAKESGSEEALERALRVAEEAARLAKRVLELAEKQGDPEVARRAVELVKRVAELLERIARESGSEEAKERAERVREEARELQERVKELREREGGWLE

SEQ ID NO: 16 - protein, 232 aa, Artificial Sequence
  Helical bundle HB5CWM3c
DPEDELKRVEKLVKEAEELLRQAKEKGSEEDLECALRTAEEAAREAKKVLEQAEKEGDPEVALRAVELVVRVAELLLRIAKESGSEEALERALRVAEEAARLAKRVLELAECQGDPEVALRAVELVVRVAELLLRIAKESGSEEALERALRVAEEAARLAKRVLELAEKQGDPEVARRAVELVKRVAELLERIARESGSEEACERAERVREEARELQERVKELREREGGWLE

SEQ ID NO: 17 - protein, 232 aa, Artificial Sequence
  Helical bundle HB5CWM4c
DPEDELCRVEKLVKEAEELLRQAKEKGSEEDLEKALRTAEEAAREAKKVLEQAEKEGDPEVALRAVELVVRVAELLLRIACESGSEEALERALRVAEEAARLAKRVLELAECQGDPEVALRAVELVVRVAELLLRIAKESGSEEALERALRVAEEAARLAKRVLELAEKQGDPEVARRAVELVKRVAELLERIARESGSEEACERAERVREEARELQERVCELREREGGWLE

SEQ ID NO: 18 - protein, 232 aa, Artificial Sequence
  Helical bundle HB5CWM5c
DPEDELKRVECLVKEAEELLRQAKEKGSEEDLECALRTAEEAAREACKVLEQAEKEGDPEVALRAVELVVRVAELLLRIACESGSEEALERALRVAEEAARLAKRVLELAECQGDPEVALRAVELVVCVAELLLRIAKESGSEEALERALCVAEEAARLACRVLELAEKQGDPEVARRAVELVCRVAELLERIARESGSEEACERAERVREEARELQERVCELREREGGWLE

SEQ ID NO: 19 - protein, 232 aa, Artificial Sequence; identical to SEQ ID NO: 18
  Helical bundle HB5CWM11c

SEQ ID NO: 20 - protein, 232 aa, Artificial Sequence
  Helical bundle HB5CWM20c
DPEDELCRVEKLVCEAEELLRQACEKGSEEDLECALRTAEEAAREACKVLEQAECEGDPEVALRAVELVVRVAELLLRIACESGSEEALECALRVAEEAARLACRVLELAECQGDPEVALRAVELVVCVAELLLRIACESGSEEALERALCVAEEAARLACRVLELAECQGDPEVARRAVELVCRVAELLECIARESGSEEACERAERVCEEARELQERVCELREREGGWLE

SEQ ID NO: 21 - protein, 484 aa, Artificial Sequence
  Helical bundle  (HB5CWM20c)2
DPEDELCRVEKLVCEAEELLRQACEKGSEEDLECALRTAEEAAREACKVLEQAECEGDPEVALRAVELVVRVAELLLRIACESGSEEALECALRVAEEAARLACRVLELAECQGDPEVALRAVELVVCVAELLLRIACESGSEEALERALCVAEEAARLACRVLELAECQGDPEVARRAVELVCRVAELLECIARESGSEEACERAERVCEEARELQERVCELREREGGWLEGGSGGSDPEDELCRVEKLVCEAEELLRQACEKGSEEDLECALRTAEEAAREACKVLEQAECEGDPEVALRAVELVVRVAELLLRIACESGSEEALECALRVAEEAARLACRVLELAECQGDPEVALRAVELVVCVAELLLRIACESGSEEALERALCVAEEAARLACRVLELAECQGDPEVARRAVELVCRVAELLECIARESGSEEACERAERVCEEARELQERVCELREREGGWLEGGGSGGSLPETGGG

SEQ ID NO: 22 - protein, 711 aa, Artificial Sequence
  Helical bundle  (HB5CWM20c)3
DPEDELCRVEKLVCEAEELLRQACEKGSEEDLECALRTAEEAAREACKVLEQAECEGDPEVALRAVELVVRVAELLLRIACESGSEEALECALRVAEEAARLACRVLELAECQGDPEVALRAVELVVCVAELLLRIACESGSEEALERALCVAEEAARLACRVLELAECQGDPEVARRAVELVCRVAELLECIARESGSEEACERAERVCEEARELQERVCELREREGGWLEGGSGGSGGSDPEDELCRVEKLVCEAEELLRQACEKGSEEDLECALRTAEEAAREACKVLEQAECEGDPEVALRAVELVVRVAELLLRIACESGSEEALECALRVAEEAARLACRVLELAECQGDPEVALRAVELVVCVAELLLRIACESGSEEALERALCVAEEAARLACRVLELAECQGDPEVARRAVELVCRVAELLECIARESGSEEACERAERVCEEARELQERVCELREREGGWLEGGSGGSDPEDELCRVEKLVCEAEELLRQACEKGSEEDLECALRTAEEAAREACKVLEQAECEGDPEVALRAVELVVRVAELLLRIACESGSEEALECALRVAEEAARLACRVLELAECQGDPEVALRAVELVVCVAELLLRIACESGSEEALERALCVAEEAARLACRVLELAECQGDPEVARRAVELVCRVAELLECIARESGSEEACERAERVCEEARELQERVCELREREGGWLE

SEQ ID NO: 23 - protein, 232 aa, Artificial Sequence
  Helical bundle HB5CWM46c
DPEDELCCVECLVCEAEELLRQACECGSEEDLECALCTAEEAACEACCVLEQAECEGDPEVALCAVELVVCVAELLLCIACESGSEEALECALCVAEEAACLACCVLELAECQGDPEVALCAVELVVCVAELLLCIACESGSEEALECALCVAEEAACLACCVLELAECQGDPEVACCAVELVCCVAELLECIACESGSEEACECAECVCEEACELQECVCELCECEGGWLE

SEQ ID NO: 24 - protein, 470 aa, Artificial Sequence
  Helical bundle (HB5CWM46c)2
DPEDELCCVECLVCEAEELLRQACECGSEEDLECALCTAEEAACEACCVLEQAECEGDPEVALCAVELVVCVAELLLCIACESGSEEALECALCVAEEAACLACCVLELAECQGDPEVALCAVELVVCVAELLLCIACESGSEEALECALCVAEEAACLACCVLELAECQGDPEVACCAVELVCCVAELLECIACESGSEEACECAECVCEEACELQECVCELCECEGGWLEGGSGGSDPEDELCCVECLVCEAEELLRQACECGSEEDLECALCTAEEAACEACCVLEQAECEGDPEVALCAVELVVCVAELLLCIACESGSEEALECALCVAEEAACLACCVLELAECQGDPEVALCAVELVVCVAELLLCIACESGSEEALECALCVAEEAACLACCVLELAECQGDPEVACCAVELVCCVAELLECIACESGSEEACECAECVCEEACELQECVCELCECEGGWLE

SEQ ID NO: 25 - protein, 241 aa, Artificial Sequence
  3HBWT
NEDDMKKLYKQMVQELEKARDRMEKLYKEMVELIQKAIELMRKIFQEVKQEVEKAIEEMKKLYDEAKKKIEQMIQQIKQGGDKQKMEELLKRAKEEMKKVKDKMEKLLEKLKQIMQEAKQKMEKLLKQLKEEMKKMKEKMEKLLKEMKQRMEEVKKKMDGDDELLEKIKKNIDDLKKIAEDLIKKAEENIKEAKKIAEQLVKRAKQLIEKAKQVAEELIKKILQLIEKAKEIAEKVLKGLE

SEQ ID NO: 26 - protein, 241 aa, Artificial Sequence; identical to SEQ ID NO: 25
  3HBM/L

SEQ ID NO: 27 - protein, 241 aa, Artificial Sequence
  3HBM/L3C
NEDDMKKLYKQMVQELCKARDRMEKLYKEMVELIQKAIELMRKIFQEVKQEVEKAIEEMKKLYDEAKKKIEQMIQQIKQGGDKQKMEELLKRAKEEMKKVKDKMEKLLEKLKQIMQEAKCKMEKLLKQLKEEMKKMKEKMEKLLKEMKQRMEEVKKKMDGDDELLEKIKKNIDDLKKIAEDLIKKAEENIKEAKKIAEQLVKRAKQLIEKAKQVAEELIKKILQLICKAKEIAEKVLKGLE

SEQ ID NO: 28 - protein, 241 aa, Artificial Sequence
  3HBM/L4C
NEDDLKKLYKQLVQELEKARDRLECLYKELVELIQKAIELLRKIFQEVKQEVEKAIEELKKLYDEAKKKIECLIQQIKQGGDKQKLEELLKRAKEELKKVKDKLEKLLEKLKQILQEAKQKLEKLLKQLKEELKKLKEKLEKLLKELKQRLEEVKKCLDGDDELLEKIKKNIDDLKKIAEDLIKKAEENIKEAKKIAEQLVKRAKQLIEKAKQVAEELIKKILCLIEKAKEIAEKVLKGLE

SEQ ID NO: 29 - protein, 241 aa, Artificial Sequence
  3HBM/L5C
NEDDLKKLYKQLVQELEKARDRLEKLYKCLVELIQKAIELLRKIFQEVKQEVEKAIEELKKLYDEAKKKIEQLIQQICQGGDKQKLEELLKRAKEELKKVKDKLEKLLEKLKQILCEAKQKLEKLLKQLKEELKKLKEKLEKLLKELKQRLEEVKKKLDGDDELLCKIKKNIDDLKKIAEDLIKKAEENIKEAKKIAEQLVKRAKQLIEKAKQVAECLIKKILQLIEKAKEIAEKVLKGLE

SEQ ID NO: 30 - protein, 241 aa, Artificial Sequence
  3HBM/L6C
NEDDMKKLYKQMVQCLEKARDRMEKLYKEMVELIQKAIELMRKIFQEVCQEVEKAIEEMKKLYDEAKKKIEQMIQQIKQGGDCQKMEELLKRAKEEMKKVKDKMEKLLEKLKQIMQEAKQKMEKLLCQLKEEMKKMKEKMEKLLKEMKQRMEEVKKKMDGDDELLEKIKKNIDDLKKIACDLIKKAEENIKEAKKIAEQLVKRAKQLIEKAKQVAECLIKKILQLIEKAKEIAEKVLKGLE

SEQ ID NO: 31 - protein, 61 aa, Artificial Sequence
  Staphylococcal
VDNKFNKEQQNAFYEILHLPNLNEEQRNAFIQSLKDDPSQSANLLAEAKKLNDAQAPKMRM

SEQ ID NO: 32 - protein, 56 aa, Artificial Sequence
  Streptococcalsp.
MTFKLIINGKTLKGEITIEAVDAAEAEKIFKQYANDYGIDGEWTYDDATKTFTVTE

(19) United States
(12) Patent Application Publication
(10) Pub. No.: US 2024/0115740 A1
(43) Pub. Date: Apr. 11, 2024

(54) PRECISE LABELING OF PROTEIN SCAFFOLDS WITH CARGO FOR USE IN BIOMEDICAL APPLICATIONS

(71) Applicants: THE TRUSTEES OF THE UNIVERSITY OF PENNSYLVANIA, Philadelphia, PA (US); AlphaThera LLC, Philadelphia (PA)

(72) Inventors: Andrew TSOURKAS, Bryn Mawr, PA (US); Feifan YU, West Windsor, NJ (US)

(73) Assignees: THE TRUSTEES OF THE UNIVERSITY OF PENNSYLVANIA, Philadelphia, PA (US); AlphaThera LLC, Philadelphia, PA (US)

(21) Appl. No.: 18/254,851

(22) PCT Filed: Nov. 30, 2021
(86) PCT No.: PCT/US21/61228

§ 371 Date: May 27, 2023

Related U.S. Application Data
(60) Provisional application No. 63/119,644, filed on Nov. 30, 2020.

(51) Int. Cl. A61K 49/00; A61K 49/16; C07K 16/28; C07K 16/32

(52) U.S. Cl. CPC A61K 49/0032; A61K 49/0056; A61K 49/16; C07K 16/2863; C07K 16/32; A61K 2123/00

(57) ABSTRACT

This invention relates to the compositions and methods for labeling antibodies and other proteins and targeting agents with a helical bundle protein that is functionalized with cargo. Cargos can include but are not limited to fluorescent dyes, haptens (e.g. biotin), contrast agents (e.g. gadolinium, radionuclides), chelated metals, therapeutic agents, sensitizers, or other small molecules. Specifically, provided herein are compositions having a helical bundle, that has been labeled at precisely defined locations with cargo, and that can be conjugated, attached or fused to an antibody or other targeting agent.

### GOVERNMENT INTEREST STATEMENT

This invention was made with government support under EB023750 awarded by the National Institutes of Health. The United States government has certain rights in the invention.

### FIELD OF THE INVENTION

This invention relates to the compositions and methods for labeling antibodies and other proteins and targeting agents with a helical bundle protein that is functionalized with cargo. Cargos can include but are not limited to fluorescent dyes, haptens (e.g. biotin), contrast agents (e.g. gadolinium, radionuclides), chelated metals, therapeutic agents, sensitizers, or other small molecules. Specifically, provided herein are compositions having a helical bundle, that has been labeled at precisely defined locations with cargo, and that can be conjugated, attached or fused to an antibody or other targeting agent.

### BACKGROUND OF THE INVENTION

In many biomedical applications (e.g., flow cytometry, enzyme-linked immunosorbent assays (ELISA), diagnostic assays, contrast-enhanced imaging, drug delivery, etc.), it is necessary to label antibodies or other targeting ligands with a desired cargo, which can include but is not limited to fluorescent dyes, haptens (e.g. biotin), contrast agents (e.g. gadolinium, radionuclides), chelated metals, therapeutic agents, sensitizers, or other small molecules. Targeting ligands can include but are not limited to proteins, small molecules, aptamers, and peptides.

The labeling of targeting ligands with cargo can be achieved by a variety of approaches. For example, the antibody or protein can be labeled directly with the cargo, whereby the cargo is covalently attached to nucleophiles such as the side chains of lysines or cysteines that are available for chemical reactions. While this approach is widely adopted due to its simplicity, it has shortcomings, such as a lack of control over which nucleophiles on the protein are labeled, potential interference of the cargo with normal protein function, and a limitation in the amount of cargo that can be attached. Too many labels can cause aggregation, precipitation, and/or loss of function. In some applications, such as labeling of antibodies/proteins with fluorescent dyes, there can also be significant self-quenching if the fluorescent dyes are too close in proximity, once attached to the protein.

To allow more control over the labeling of antibodies or proteins, nucleophiles or unnatural amino acids can be incorporated into the coding sequence of the protein, to act as chemical handles and allow for site-specific labeling with cargo. In particular, it may be possible to limit the attachment of cargo to these unique chemical handles that have been introduced into the protein backbone. However, many of the same limitations may still exist. For example, the amount of unique chemical handles that can be introduced, without interfering with protein function is limited, thus limiting the amount of payload that can be attached.

In some assays, the amount of cargo that can be attached to a ‘primary’ antibody can be significantly increased with the use of secondary antibodies. Secondary antibodies can be labeled with the desired cargo and selected to bind the primary antibody. Since multiple secondary antibodies can bind a single primary antibody, the amount of cargo that can be bound to the primary antibody can be quite extensive. However, the use of secondary antibodies is generally limited to in vitro (research or diagnostic) assays. Moreover, binding is non-covalent, which can lead to dissociation of the secondary antibody from the primary antibody under some circumstances. In addition, the use of secondary antibodies can limit multiplexing capabilities in applications such as flow cytometry, since unique primary and secondary antibodies must be carefully paired. The use of secondary antibodies also requires additional incubation and washing steps, in contrast to assays in which the primary antibody is labeled. Thus, these assays are more time consuming.

An alternative approach for labeling of antibodies/proteins with cargo involves the use of nanoparticles. Nanoparticles can be loaded/functionalized with high payloads of cargo; however, nanoparticles are generally much bigger than antibodies/proteins, are generally not precisely defined structures, can diffuse very slow due to their large size, can exhibit a high level of non-specific interactions, and for in vivo applications can exhibit poor tissue penetration. Therefore, nanoparticles are not suitable or not desirable for many biomedical applications.

Accordingly, there exists a need to develop a modular approach to efficiently label antibodies, proteins or other targeting ligands with a large number of cargo, without interfering with normal function.

### SUMMARY OF THE INVENTION

In one aspect, provided herein are protein scaffolds and compositions thereof comprising: (i) a helical bundle having a plurality of chemical handles and (ii) cargo, wherein the chemical handles have been labeled with the cargo.

In one aspect, provided herein are protein scaffolds and compositions thereof, comprising: (i) a helical bundle having a plurality of chemical handles at defined locations and (ii) cargo, wherein the chemical handles have been labeled with the cargo. In some embodiments, the chemical handles have been introduced at high density on the surface of the helical bundle. In some embodiments, the chemical handles have been labeled with fluorescent dyes, and the chemical handles are spaced so as to limit the quenching of the fluorescent dyes. In one aspect, provided herein are protein scaffolds and compositions thereof comprising: a plurality of helical bundles in tandem labeled with cargo, wherein each helical bundle has a plurality of chemical handles, and wherein the chemical handles have been labeled with the cargo.

In one aspect, provided herein are protein scaffolds and compositions thereof comprising: a helical bundle, wherein the helical bundle has been designed to include a plurality of a first chemical handle, and a single second chemical handle that is distinct from the first, wherein the first chemical handle can be a lysine, a cysteine, an unnatural amino acid or combination thereof, wherein the second chemical handle can be a lysine, a cysteine, an amine, a thiol, an unnatural amino acid, a click-chemistry group, a thiol-reactive moiety, or an amine-reactive moiety, and wherein the first chemical handle is labeled with cargo, and wherein the second chemical handle allows for the attachment of said helical bundle to a protein, nucleic acid, small molecule, particle, or surface.

In one aspect, provided herein are protein scaffolds and compositions thereof comprising: a helical bundle that has been operably linked to a moiety selected from a protein, a nucleic acid, a polymer, a lipid, a small molecule or a combination thereof, and wherein the helical bundle has been labeled with a plurality of cargo. In some embodiments, the moiety is a targeting ligand. In one embodiment, the moiety is an antibody-binding domain (AbBD). In some embodiments, the antibody binding domain is operably linked to a photoreactive amino acid group, for example, benzoylphenylalanine (BPA) resulting in a photoreactive antibody binding domain (pAbBD).

In one aspect, provided herein are methods for imaging and/or detecting cells (e.g., tumor cells) in vitro, comprising: (a) contacting the cells with a protein scaffold described herein that is operably linked to a targeting ligand, wherein the targeting ligand binds to a component of the cells; and imaging and/or detecting the cells by visualizing and/or detecting the cargo of the protein scaffold.

In one aspect, provided herein are methods for imaging cells or tissue (e.g., tumor cells or tumor tissue) in a subject, comprising: (a) administering to the subject a protein scaffold described herein that is operably linked to a targeting ligand, wherein the targeting ligand binds to a component of the cells or tissue; and visualizing the cells or tissue by detecting the cargo of the protein scaffold.

In one aspect, provided herein are methods for intraoperative optical image-guided surgery of a tumor in a subject, comprising: (a) administering to the subject a protein scaffold described herein that is operably linked to a targeting ligand, wherein the targeting ligand binds to a surface of cancers cells of the tumor; visualizing the tumor and delineating intraoperative margins thereof during the surgery by detecting the cargo of the protein scaffold; and (c) resecting the tumor at or near the delineated intraoperative margins thereof.

Other features and advantages of this invention will become apparent from the following detailed description examples and figures. It should be understood, however, that the detailed description and the specific examples while indicating preferred embodiments are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of this disclosure, the inventions of which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIG. 1. Illustration of various helical bundle structures. A helical bundle is a protein composed of two or more alpha helices, represented here as colored cylinders. The alpha helices are usually nearly parallel or antiparallel to each other. Multiple alpha helices can also be fused together in tandem.

FIG. 2. Different views of a representative 4-helical bundle. From Huang et al. High thermodynamic stability of parametrically designed helical bundles. Science. 2014; 346 (6208): 481-485.

FIG. 3. Illustration of a helical bundle with lysine residues highlighted with red circles. The green cylinders represent alpha helices. The lysines can subsequently be labeled with cargo. Cargo can include but are not limited to fluorescent dyes, haptens (e.g. biotin), contrast agents (e.g. gadolinium, radionuclides), chelated metals, therapeutic agents, sensitizers, small molecules, or combinations thereof.

FIG. 4. Illustration of helical bundle designs with cysteine residues, highlighted with red circles, introduced at precisely defined locations. The green cylinders represent alpha helices. The cysteines can subsequently be labeled with thiol-reactive cargos. The designs were named 4a, 4b, 6a, 6b, 8a and 8b.

FIG. 5. PyMOL views of helical bundle HB1P68 (blue) with 6 Cysteine residues (shown in yellow) introduced into the structure.

FIG. 6. PyMOL view of helical bundle HB5CWM (green) with 20 Cysteines (shown in blue) residues introduced into the structure (i.e., HB5CWM20c). A helical bundle was also created by fusing together two HB SCWM20C domains in tandem (HB5CWM20c)2.

FIG. 7. Illustration of a process that is used to create a helical bundle with cysteine residues introduced at precisely defined locations and an azide at or near the c-terminus. The helical bundle, with cysteines at defined locations, is expressed in series with a sortase recognition motif, sortase, and an affinity tag (His tag). The expressed protein is captured on an affinity matrix (e.g. IMAC Ni²⁺ resin). Addition of the peptide GGG-azide and calcium leads to the sortase mediated ligation of the GGG-azide peptide to the sortase recognition motif. This also results in the release of the helical bundle, now with a c-terminal azide, from the affinity matrix. This process is called Sortase-tag expressed protein ligation (STEPL). The cysteines are subsequently labeled with the desired cargo via a thiol-maleimide reaction.

FIG. 8. An illustration of a Her2-targeted affibody (light blue) that is genetically fused to a helical bundle (green). The helical bundle has multiple cysteines introduced into the sequence, shown in red. The cysteines were optimally placed to minimize quenching when labeled with fluorescent dyes. The Her2Affibody is known to strongly binding to Her2 protein which is breast cancer biomarker, while the helical bundle was labeled with the near infrared imaging dye, maleimide-ICG. In this picture, HER2 protein with different extracellucar domains were shown together with Her2 Affibody (3MZW.pdb) and helical bundle (4OUS.pdb). As an alternative to a fluorescent dye, the cysteines can also be labeled with other cargo including but not limited to haptens (e.g. biotin), contrast agents (e.g. gadolinium, radionuclides), chelated metals, therapeutic agents, sensitizers, small molecules, or combinations thereof.

FIGS. 9A-9B. (A) Illustration of a photoreactive antibody binding domain (pAbBD, shown in yellow) that has been genetically fused with a helical bundle (green). The pAbBD has been photocrosslinked to an antibody (blue). The helical bundle has multiple cysteines introduced into the sequence, shown in red. The cysteines can be labeled with cargo that includes but is not limited to fluorescent dyes, haptens (e.g. biotin), contrast agents (e.g. gadolinium, radionuclides), chelated metals, therapeutic agents, sensitizers, small molecules, or combinations thereof. The pAbBDs-helical bundle fusion protein allows the helical bundle to be site-specifically attached to an antibody. If the helical bundle is first labeled with multiple cargo, the pAbBD effectively labels the antibody with this cargo. One of the advantages of using the helical bundle platform is that more cargo can be attached to the antibody via the HB, without fear of interfering with normal antibody function. The location of the cargo on the helical bundle can also be controlled. Thus, the spacing can be adjusted to minimize fluorescent quenching when the cysteines are labeled with fluorescent dyes. (B) Illustration of an antibody that has been labeled using a traditional approach, whereby some of the Lysines (amines, shown in red) on the antibody (blue) are labeled. Direct labeling of antibody with cargo can interfere with normal antibody function, since the cargo may reside in or near the antibody-binding site. Accordingly, this approach also has strict limitations on the amount of cargo that can be added. Direct labeling of the lysines on the antibody also does not allow for control over the spacing between the attached cargo. Therefore, when antibodies are directly labeled with fluorescent dyes, there can be significant fluorescent quenching.

FIG. 10. Cetuximab (Cetu), an anti-EGFR antibody, or Rituximab (Ritu), an anti-CD20 antibody, were labeled with different pAbBD-HB488 constructs and analyzed by SDS-PAGE. pAbBD-HB488 is a photoreactive antibody-binding domain-helical bundle fusion protein that has been labeled with a fluorescent dye, with an excitation wavelength of ˜488 nm. A) SDS-PAGE gel stained with SimpleBule™ SafeStain. B) SDS-PAGE gel under UV exposure. The samples include: 1. Cetu-pAbBD-HB4a488; 2. Ritu-pAbBD-HB4a488; 3. Cetu-pAbBD-HB4b488; 4. Ritu-pAbBD-HB4b488; 4. Cetu-pAbBD-HB6a488; 5. Ritu-pAbBD-HB6a488; 6. Cetu-pAbBD-HB6b488; 7. Ritu-pAbBD-HB6b488; 8. Cetu-pAbBD-HB8a488; 9. Ritu-pAbBD-HB8a488; 10. Cetu-pAbBD-HB8b488 and 11. Ritu-pAbBD-HB8b488. The notation HB4a, HB4b, HB6a, HB6b, HB8a, HB8b represent helical bundles with 4, 6, or 8 cysteines at various locations. The cysteines were labeled with a fluorescent dye with an excitation wavelength of ˜488 nm.

FIG. 11. SDS-PAGE gel of pAbBD-HB6a fusion protein (lane 1) and pAbBD-HB6a 3ME (lane 2), which includes additional mutations that prevent the expression of truncation products. pAbBD-4HB 3ME includes the 3 mutations M73E. M84E and M101E, which eliminates truncation products.

FIG. 12. SDS-PAGE of Cetuximab (Cetu) and Rituximab (Ritu) antibodies labeled with different pAbBD-HB488 constructs. pAbBD-HB488 is a photoreactive antibody-binding domain-helical bundle fusion protein that has been labeled with a fluorescent dye, with an excitation wavelength of ˜488 nm (A) The gel was stained with SimpleBule™ SafeStain or (B) visualized under UV exposure. The samples include 1. Cetu-pAbBD-HB6a, 2. Cetu-pAbBD-HB6a3ME, 3. Cetu-pAbBD-HB6c3ME, 4. Cetu-pAbBD-HB5CWM3c, 5. Cetu-pAbBD-HB5CWM5c, 6. Cetu-pAbBD-HB5CWM11c, 7. Ritu-pAbBD-HB6a, 8. Ritu-pAbBD-HB6a3ME, 9. Ritu-pAbBD-HB6c3ME, 10. Ritu-pAbBD-HB5CWM3c, 11. Ritu-pAbBD-HB5CWM5c, 12. Ritu-pAbBD-HB5CWM11c. The cysteines in each construct were labeled with a fluorescent dye, with an excitation wavelength of ˜488 nm. HC represents the heavy chain of the antibody.

FIG. 13. SDS-PAGE of anti-BSA rabbit antibody after photocrosslinking with different pAbBD-HB fusion proteins that were labeled with biotin. The gel was stained with SimpleBule™ SafeStain. Lane 1: Anti-BSA rabbit antibody conjugated with pAbBD-HB6a-Biotin; Lane 2: Anti-BSA rabbit antibody conjugated with pAbBD-HB5CWM20c-Biotin; Lane 3: Anti-BSA rabbit antibody conjugated with pAbBD-(HB5CWM20c)2-Biotin and Lane 4: Rabbit isotype control antibody conjugated with pAbBD-(HB5CWM20c)2-Biotin. The SDS-PAGE gel shows antibody heavy chains were site-specifically and efficient labeled with pAbBD-HB-biotin constructs.

FIGS. 14A-14C. Illustration of immunostaining of A549 cells via three different methods. (A) A549 cells are labeled with a primary antibody conjugated to pAbBD-HB488. (B) A549 cells are first labeled with a primary antibody. Then a secondary antibody that has been labeled with a fluorescent dye is added. The lysines on the secondary antibody are labeled with an amine-reactive dye (eg. NHS-488). (C) A549 cells are labeled with a primary antibody that has been directly labeled with fluorescent dyes. The lysines on the primary antibody are labeled with an amine-reactive dye (e.g. NHS-488).

FIGS. 15A-15H. Flow cytometry data of EGFR-positive cells labeled with an anti-EGFR primary antibody (Cetuximab) conjugated to various pAbBD-HB488 conjugates or detected with a 2^{nd} goat anti-human FITC antibody. (A) The brown curve indicates fluorescent intensity of cells labeled with Cetuximab-pAbBD-HB4a-488 (Fluorescein), while the red curve and black curves indicate fluorescent signals of the non-targeted control, Rituximab-pAbBD-HB4a-488 (Fluorescein) and A549 cells only, respectively. FIGS. 15B-15F are analogous flow cytometry data whereby antibodies were labeled with pAbBD-HB4b-488, pAbBD-HB6a-488, pAbBD-HB6b-488, pAbBD-HB8-488a and pAbBD-HB8b-488 accordingly. Similar flow cytometry results were observed for the helical bundles of SEQ ID NOs: 25-30 (data not shown). (G) A549 cells were incubated with unlabeled Cetuximab or Rituximab and after wash, goat anti-human IgG-FITC antibody was applied. The pink curve showed the fluorescent signal of Cetuximab plus 2^{nd} antibody while the orange and black curve showed the fluorescent signals of Rituximab combined with 2^{nd} antibody and A549 cells only. (H) Overlap of all curves.

FIGS. 16A-16C. Flow cytometry data of EGFR-positive cells labeled with Cetuximab (Citu), an anti-EGFR primary antibody, conjugated to pAbBD-HB6a-488 (Fluorescein) or detected with a 2^{nd} goat anti-human FITC antibody. (A) The darker blue curve shows the fluorescent signals of cells labeled with Cetuximab-pAbBD-HB6a-488 (Fluorescein), while the light blue curve and black curves shows fluorescent signals of cells labeled with the negative control Rituximab (Ritu)-pAbBD-HB6a-488 (Fluorescein) and A549 cells only, respectively. (B) A549 cells were incubated with unlabeled Cetuximab or Rituximab and after wash, goat anti-human IgG-FITC antibody was applied. The pink curve shows the fluorescent signal of cells labeled with Cetuximab plus 2^{nd} antibody while the orange and black curve show the fluorescent signals of cells labeled with Rituximab combined with 2^{nd} antibody and A549 cells only, respectively. (C) The overlap of all curves indicated similar fluorescent signals.

FIG. 17A-17D. Flow cytometry data of EGFR-positive A549 cells labeled with Cetuximab (Citu), an anti-EGFR primary antibody, conjugated to pAbBD-HB6a-488 (Alexa488) or detected with a 2nd goat anti-human Alexa488 antibody. (A) The blue curve shows the fluorescent signals of cells labeled with Cetuximab-pAbBD-HB6a-488 (Alexa488), while the pink curve and black curves shows fluorescent signals of cells labeled with the negative control Rituximab (Ritu)-pAbBD-HB6a-488 (Alexa488) and A549 cells only, respectively. (B) A549 cells were incubated with unlabeled Cetuximab or Rituximab and after wash, goat anti-human IgG-Alexa488 antibody was applied. The purple curve shows the fluorescent signal of cells labeled with Cetuximab plus 2^{nd} antibody while the blue and black curve show the fluorescent signals of cells labeled with Rituximab combined with 2^{nd} antibody and A549 cells only, respectively. (C) A549 cells were labeled with Cetuximab or Rituximab antibodies that were labeled directly with Alexa488. These antibodies were labeled using a 20:1 reaction ratio of NHS-Alexa488 to antibody. (D) The overlap of all curves indicated different fluorescent signals of method A, B and C.

FIG. 18. Flow cytometry data of EGFR-positive A549 cells after labeling with an anti-EGFR primary antibody (Cetuximab) that was labeled with NHS ester Alexa488 at various reaction ratios. The brown, green, purple and blue curves show the fluorescent intensity of cells that were labeled with Cetuximab that was reacted with NHS ester Alexa 488 at ratios of 1:5, 1:10, 1:20 and 1:40. The orange curve shows the fluorescent signals of A549 cells labeled with Rituximab (labeled with NHS ester Alexa488 using 1:20 ratio of Rituximab:NHS-Alexa488) and black curve shows A549 cells only.

FIG. 19. EGFR-positive A549 cells were incubated with fluorescently labeled anti-EGFR antibodies (Cetuximab) and analyzed by flow-cytometry. Antibodies were either labeled with amine-reactive dyes from Competitor A (excitation=488 nm) or with pAbBD-HB488. For the amine-reactive dyes, the dye:antibody ratio was varied from 5:1 to 40:1 to find the conditions that led to a maximum fluorescent intensity. pAbBD-HB488 led to signals that were >6.5-times brighter than the brightest signal produced from Competitor A's equivalent product.

FIG. 20. Cetuximab (anti-EGFR antibody) was photocrossinked to a pAbBD with a single C-termimal Alexa488 dye (attached via a c-terminal cysteine) or pAbBD-HB with 6 Cysteines that were labeled with maleimide-Alexa488. A flow cytometry assay showed that the antibodies labeled with pAbBD-HB-Alexa488 led to a significant fluorescent shift in the fluorescence of labeled A549 cells (purple curve), compared to when pAbBDs with a single fluorescent label were used (green curve). Unlabeled cells are indicated by the brown curve.

FIGS. 21A-21C. Illustrations of enzyme-linked immunosorbent assays (ELISA) being performed using three different approaches. (A) Antigen (BSA) is labeled with a primary anti-BSA antibody the has been photocrosslinked to pAbBD-HB-biotin (shown in purple). The biotin is then labeled with a streptavidin (SA)-horseradish peroxidase (HRP) conjugate. (B) Antigen is labeled with a primary antibody (unconjugated). This primary antibody is then labeled with a secondary (2^{nd}) antibody that is conjugated to HRP. (C) Antigen is labeled with a biotinylated primary antibody, which is subsequently labeled with a SA-HRP conjugate. The lysines on the primary antibody were labeled with NHS-biotin. In all cases, the presence of HRP can be detected upon the addition of HRP substrates.

FIG. 22. Enzyme-linked immunosorbent assays (ELISA) were performed using three different approaches. In one approach, the primary antibody was photo-crosslinked with pAbBD-HB-Biotin. In a second approach, the primary antibody was labeled with NHS ester Biotin. In the third approach, the primary antibody was labeled with a secondary (2^{nd}) HRP conjugated antibody. Various pAbBD-HB-biotin constructs were tested, whereby helical bundles differed in the number and location of cysteines. The cysteines were labeled with Maleimdie-PEG2-Biotin. The polyclonal anti-BSA rabbit antibody was photo-crosslinked to each pAbBD-HB-biotin. Then the conjugated antibody was added into a precoated BSA ELISA plate. Streptavidin-HRP was added and then the HRP substrate. For comparison, goat anti-BSA antibody directly labeled with NHS ester biotin was also tested. In addition, the polyclonal anti-BSA rabbit antibody in combination with a 2^{nd} goat anti-rabbit HRP was tested.

FIGS. 23A-23B. Enzyme-linked immunosorbent assays (ELISA) for the detection of (A) BSA or (B) IL-6 antigens were performed using three different approaches, (i) the primary antibody was photo-crosslinked with pAbBD-HB-Biotin and then added to microplate wells coated with the antigen; (ii) the primary antibody was added to microplate wells coated with antigen and then labeled with a 2^{nd} HRP conjugated antibody; or (iii) the primary antibody was labeled with NHS-Biotin and then added to microplate wells coated with the antigen. Samples that included biotinylated primary antibody were further labeled with streptavidin-HRP conjugates. All samples were subsequently incubated with HRP substrate, for detection. The samples with pAbBD-HB-biotin exhibited the highest ELISA sensitivity for both BSA and IL-6 detection.

FIGS. 24A-24C. Flow cytometry data of A549 cells that had EGFR expression detected via two different approaches. (A) In the first approach, the anti-EGFR antibody Cetuximab (Citu) was first photocrosslinked with pAbBD-HB-Biotin. A549 cells were then incubated with the Cetu-pAbBD-biotin conjugate, followed by Streptavidin PE (blude curve). PE fluorescence was detected by flow cytometry. The anti-CD20 antibody Rituximab (Ritu), which was also photocrosslinked with pAbBD-HB-biotin, was used as a negative control (orange curve). The flow cytogram of unlabeled cells is also shown (black curve). (B) In the second approach, cells were labeled with Cetuximab (unmodified) first, followed by the addition of a 2^{nd} goat anti-human PE antibody (brown curve). A negative control using the anti-CD20 antibody Rituximab as the primary antibody was also performed (purple curve). The flow cytogram of unlabeled cells is also shown (black curve). (C) Overlap of all curves.

FIG. 25. SDS-PAGE of Cetuximab alone and after photo-crosslinking with pAbBD-(HB5CWM20c)2-DOTA-Nd150. pAbBD-(HB5CWM20c)2 was first reduced with TCEP and then labeled with maleimide-DOTA-Nd150. The gel was stained with SimpleBlue™ SafeStain. Lane 1: 0.5 μg Cetuximab and Lane 2: 0.5 μg Cetuximab conjugated with pAbBD-(HB5CWM20c)2-DOTA-Nd150. The SDS-PAGE gel shows that antibody heavy chains were efficiently labeled with pAbBD-(HB5CWM20c)2-DOTA-Nd150.

FIG. 26. CyTOF data of A549 cells following incubation with antibodies labeled with pAbBD-(HB5CWM20c)2-DOTA-Nd150. Cetuximab or Rituximab were site-specifically conjugated with pAbBD-HB5CWM20c)2-DOTA-Nd150, and then 5 ug/ml conjugated antibody was incubated with A549 (EGFR+) cells. After washing three times with PBS buffer, the cells were fixed and Ir intercalator stained. A. The red curve shows Nd150 signals from Cetuximab-pAbBD-(HB5CWM20c)2-DOTA-Nd150. B. The blue curve shows Nd150 signals from Rituximab-(HB5CWM20c)2-DOTA-Nd150. C. The overlap of A and B curves. The S/N of mean was ˜200 (361/1.78).

FIG. 27. SDS-PAGE of Trastuzumab (anti-Her2 antibody) alone of after labeling with DOTA-Gd160, using three different approaches. In the first approach, Trastuzumab was labeled with pAbBD-(HB5CWM20c)2-DOTA-Gd160. pAbBD-(HB5CWM20c)2 was first reduced with TCEP and then labeled with maleimide-DOTA-Gd160. In the second approach, Transtuzumab was labeled with polymer-Gd160 conjugation kit (competitor A). In the third approach, Trastuzumab was labeled with an amine-reactive Gd160 metal conjugation kit (competitor B). The gel was stained with SimpleBlue™ SafeStain. Lane 1: 0.5 μg Trastuzumab; Lane 2: 0.5 μg Trastuzumab labeled with pAbBD-(HB5CWM20c)2-DOTA-Gd160; Lane 3: 0.5 μg Trastuzumab labeled with polymer-Gd160 conjugation kit and Lane 4: 0.5 μg Trastuzmab labeled with amine-reactive Gd160 metal conjugation kit. pAbBD-(HB5CWM20c)2 is the only method that results in a uniformly labeled antibody heavy chain.

FIG. 28. CyTOF data of Her2-positive SKBR3 cells following incubation with antibodies labeled with pAbBD-(HB5CWM20c)2-DOTA-Gd160, polymer-Gd160 conjugation kit (competitor A), or amine-reactive Gd-160 conjugation kit (competitor B). A. CyTOF signal obtained by using Trastuzumab conjugated with polymer-Gd160 (red) or Rituximab conjugated with polymer-Gd160 (blue); B. CyTOF signal obtained by using Trastuzumab conjugated with pAbBD-(HB5CWM20c)2-DOTA-Gd160 (red) or Rituximab conjugated with pAbBD-(HB5CWM20c)2-DOTA-Gd160 (blue); C. CyTOF signal obtained by using Trastuzumab conjugated with amine-reactive Gd-160 conjugation kit (red) or Rituximab conjugated with amine-reactive Gd-160 conjugation kit (blue).

FIGS. 29A-29B. Flow Cytometry analysis of Her2-positive cells labeled with Her2-targeted Affibodies (Affi342) that have been labeled with a single dye at the c-terminus or fused with a helical bundle that has been labeled with multiple dyes. As proof of concept, a Maleimide-Alexa488 dye was used as the fluorescent label. Affi342 with a cysteine near its c-terminus was labeled with a single dye (Affi342-C-Alexa488), while and affi342-HB fusion protein was labeled with 6 dyes, owing to the presence of 6 cysteines that were introduced at specific positions within the HB. (A) Flow cytometric analysis of Her2-positive T6-17 cells labeled with Affi342-HB-Alexa488 (pink curve), Affi342-C-Alexa488 (blue curve) and T6-17 cells alone (brown curve). (B) Flow cytometric analysis of Her2-negative A549 cells labeled with Affi342-HB-Alexa488 (red curve), Affi342-C-Alexa488 (brown curve) and T6-17 cells alone (black curve).

FIGS. 30A-30B. (A) Intraoperative and (B) ex-vivo brightfield and overlay fluorescent image of invasive breast cancer in a human patient.

FIGS. 31A-31B. (A) Schematic of Her2-targeted helical bundle. Locations of cysteines that can be labeled with dye are highlighted in red. (B) Flow cytometry histogram of EGFR-positive cells labeled with an anti-EGFR antibody randomly conjugated with Alexa488 or an Anti-EGFR antibody conjugated with helical bundle-Alexa488.

FIG. 32. Schematic of S0456-maleimide.

FIGS. 33A-33B. (A) Schematic of Her2-targeted helical bundle. Locations of cysteines are highlighted in red. (B) Flow cytometry histogram of Her2-positive cells labeled with a Her2-targeted affibody labeled at the c-terminus with a single Alexa488 (Her2-Alexa488) or a Her2-targeted 4HB labeled with Alexa488.

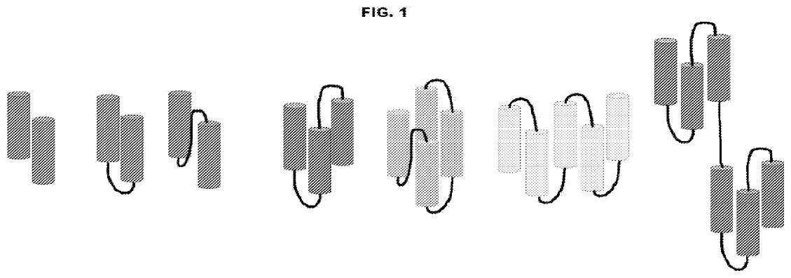

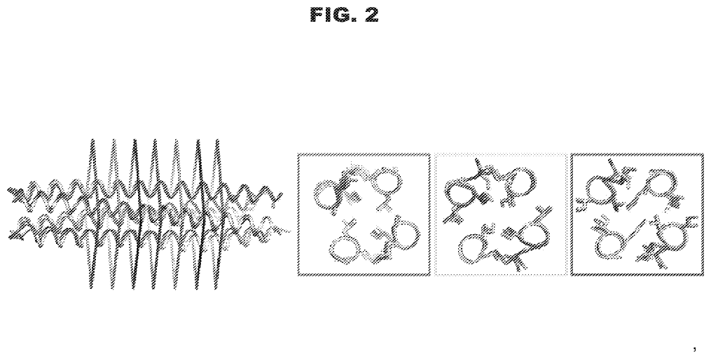

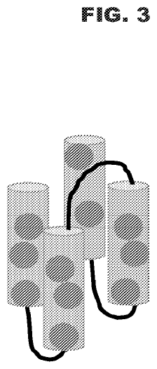

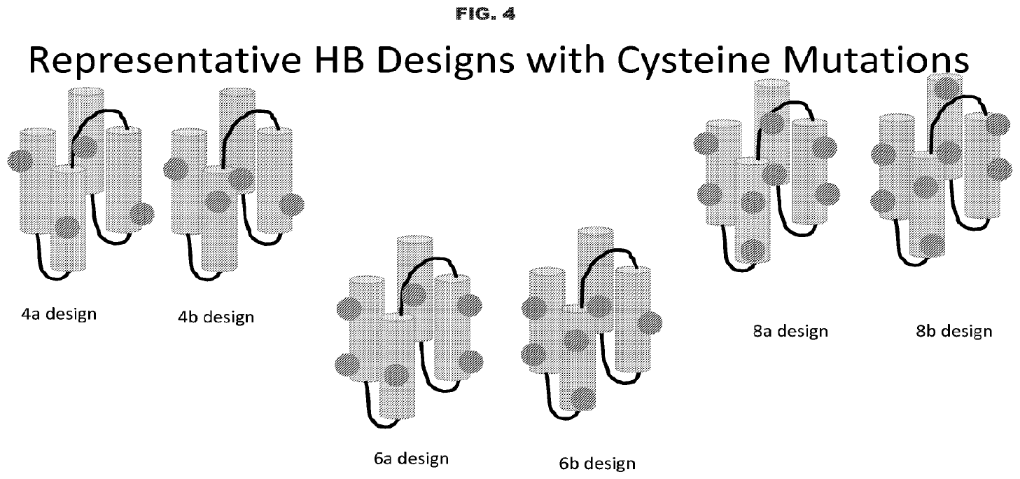

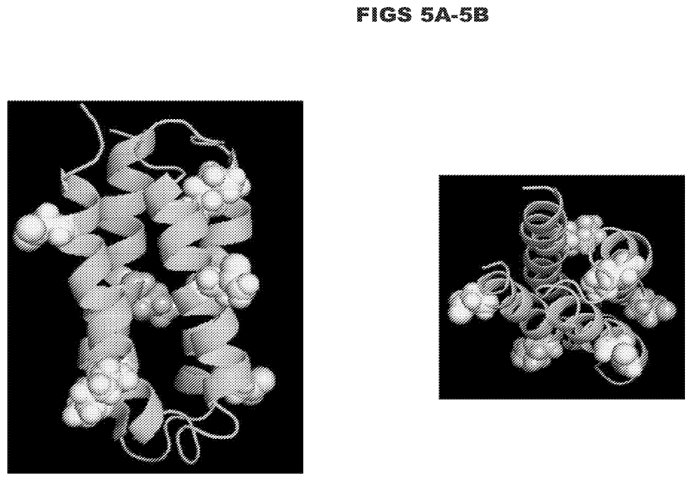

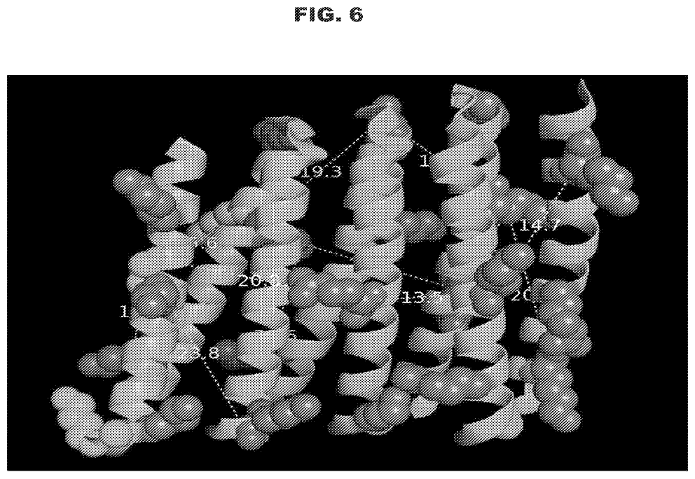

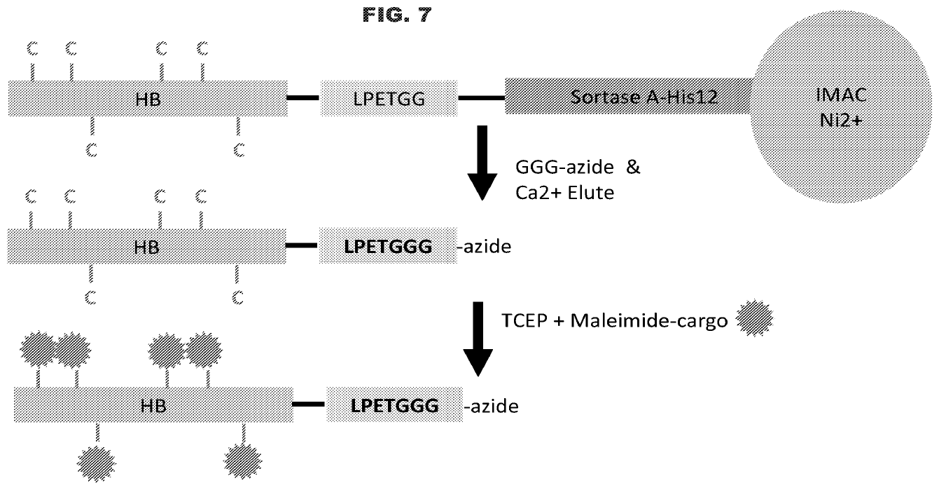

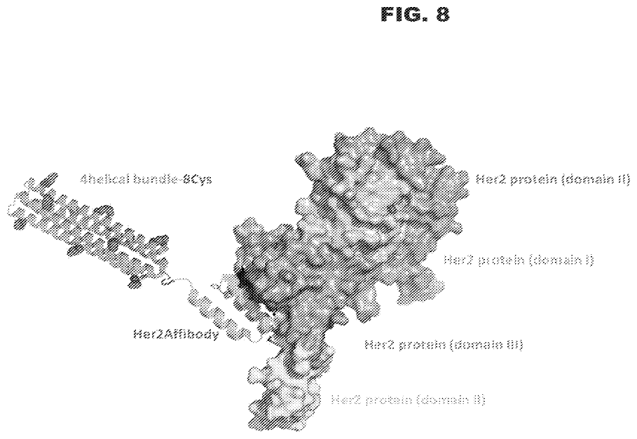

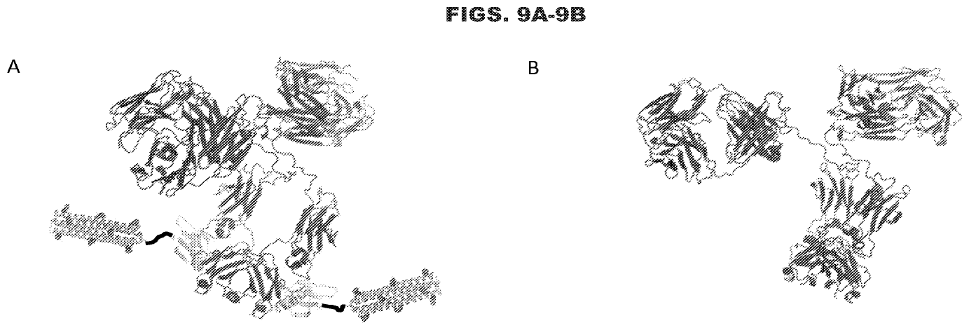

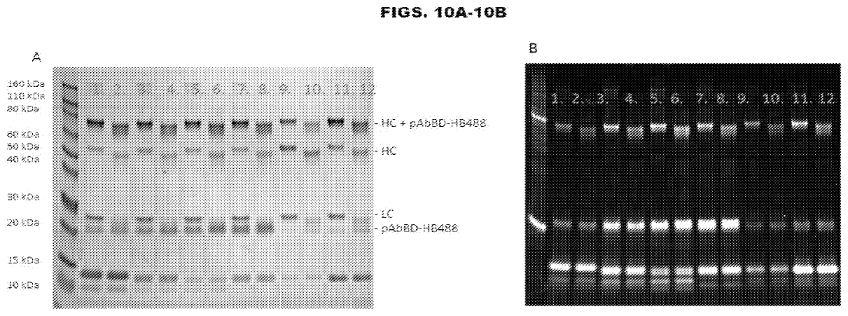

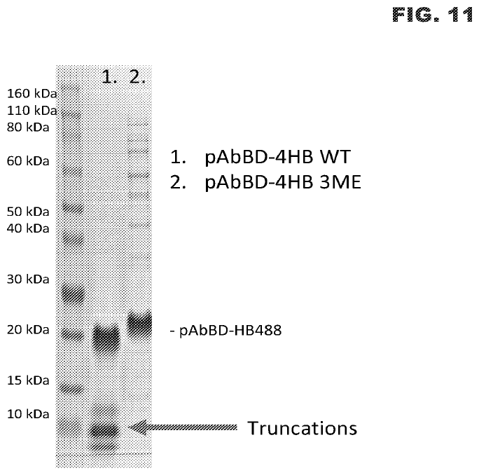

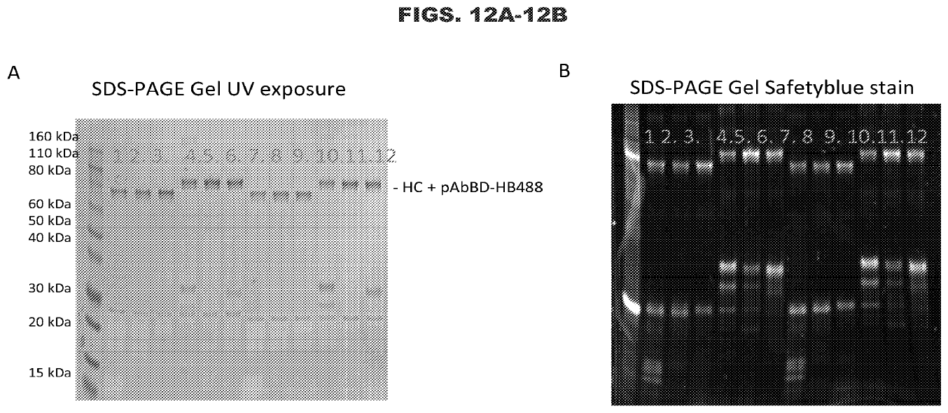

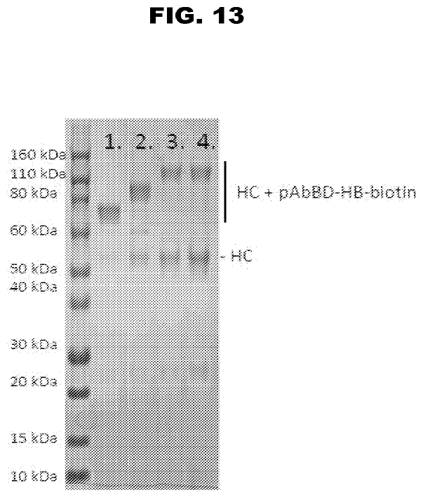

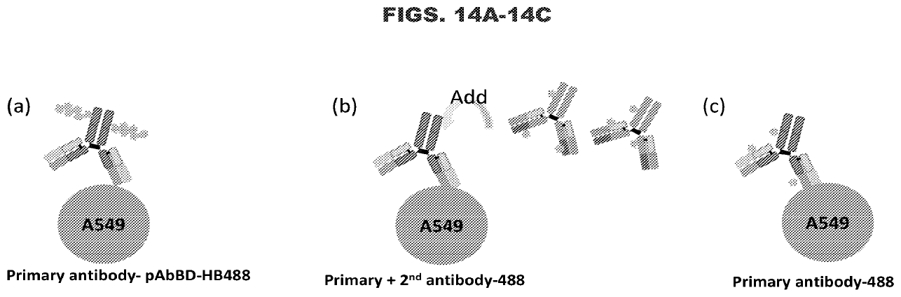

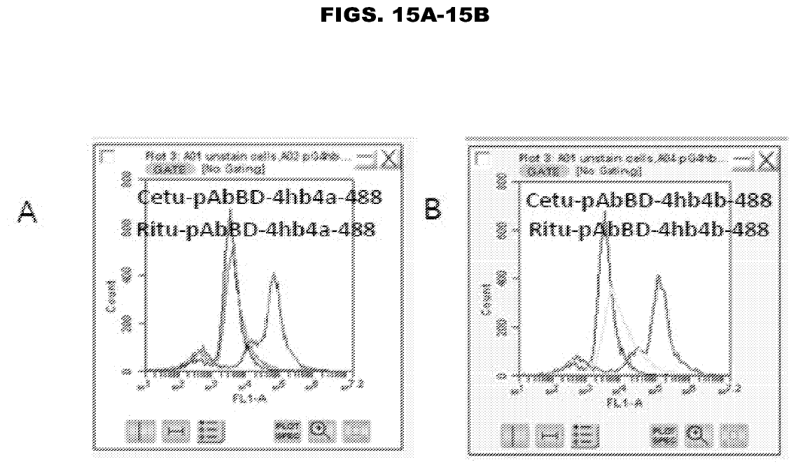

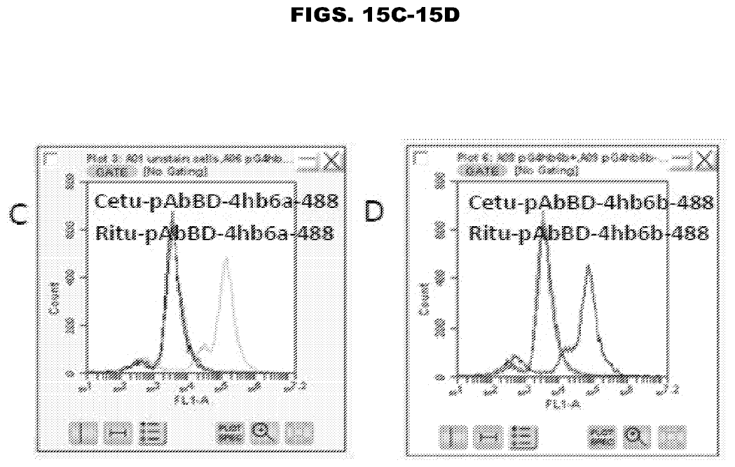

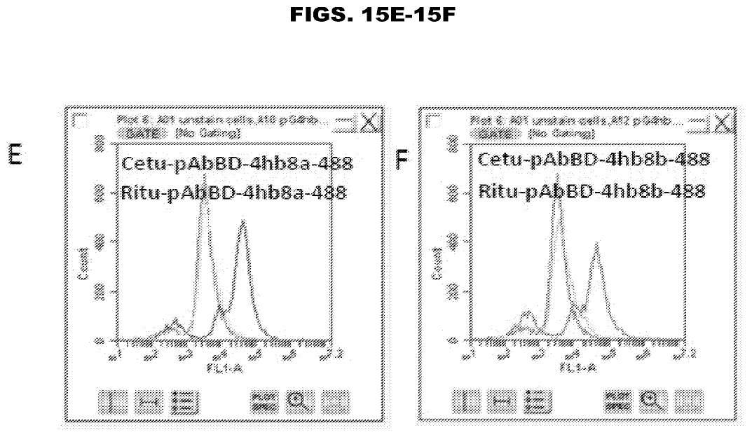

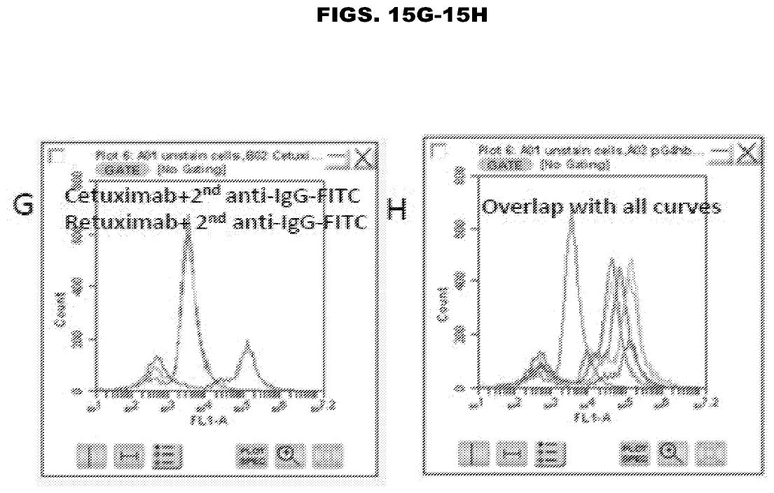

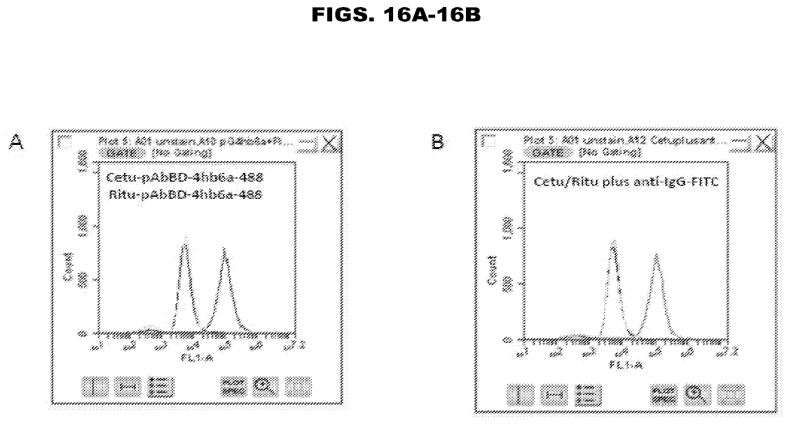

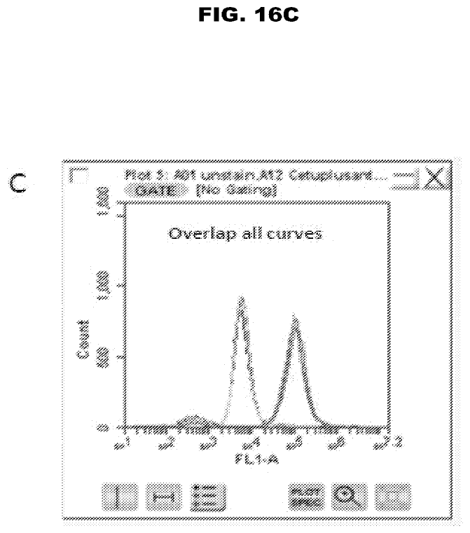

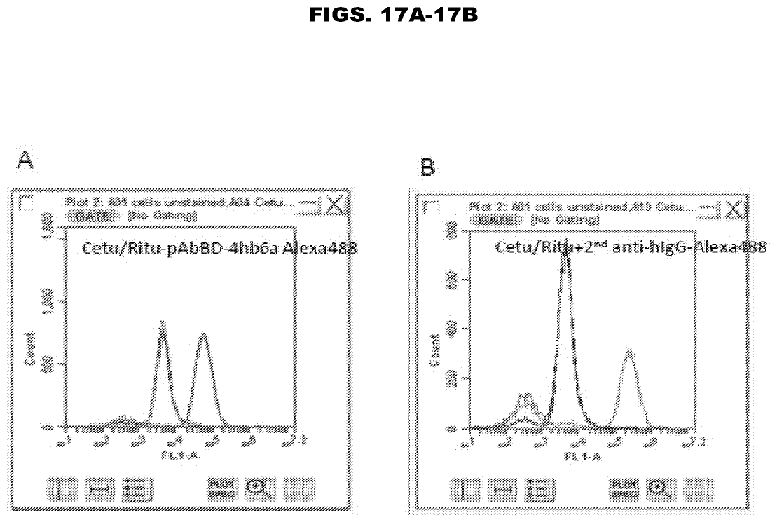

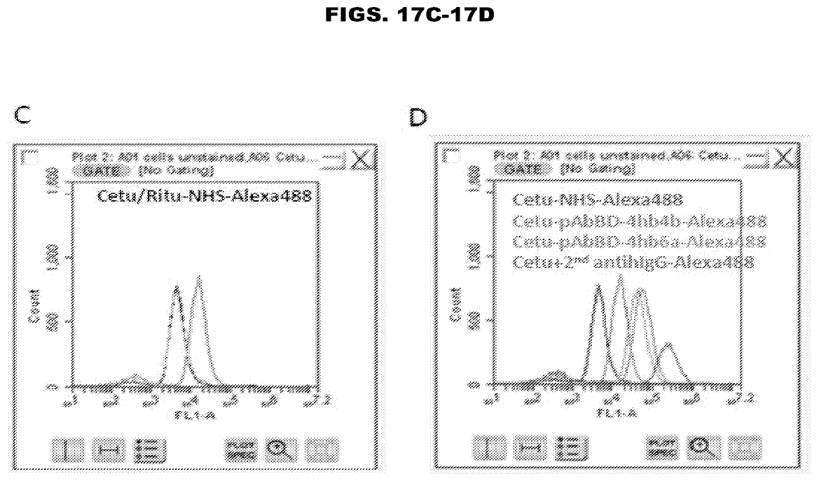

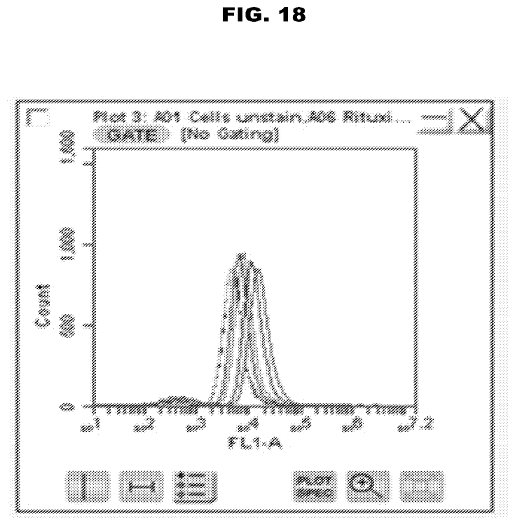

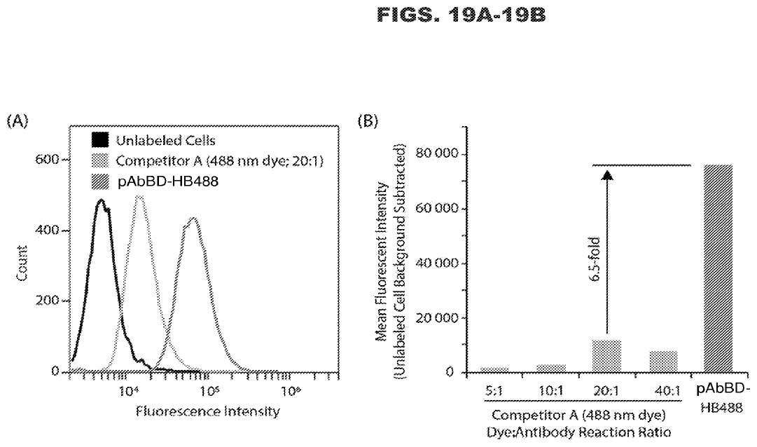

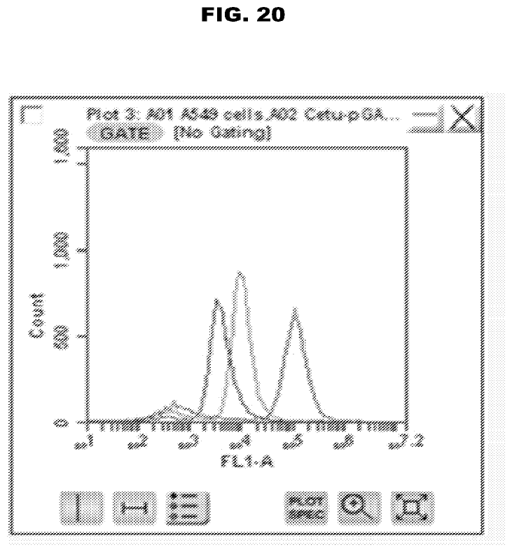

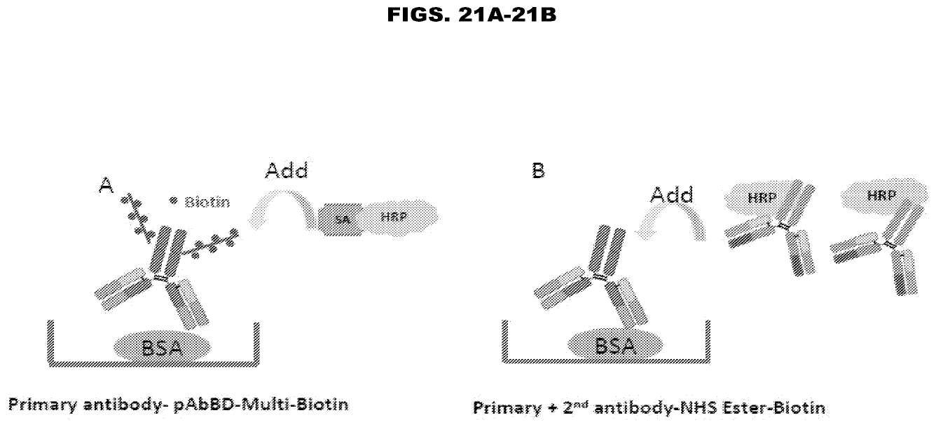

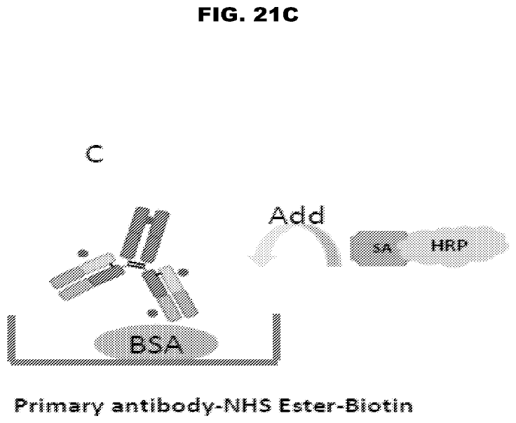

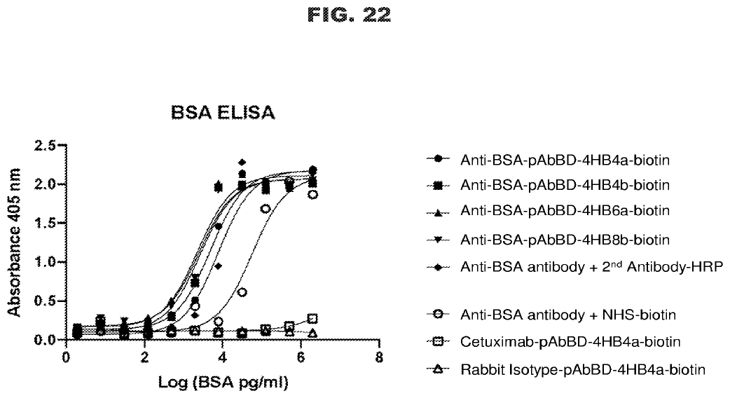

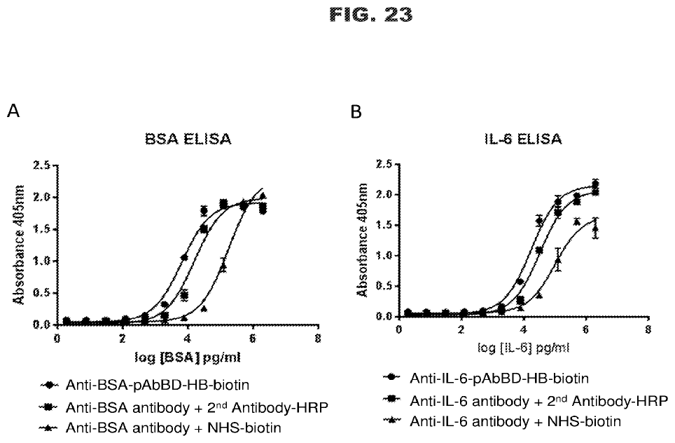

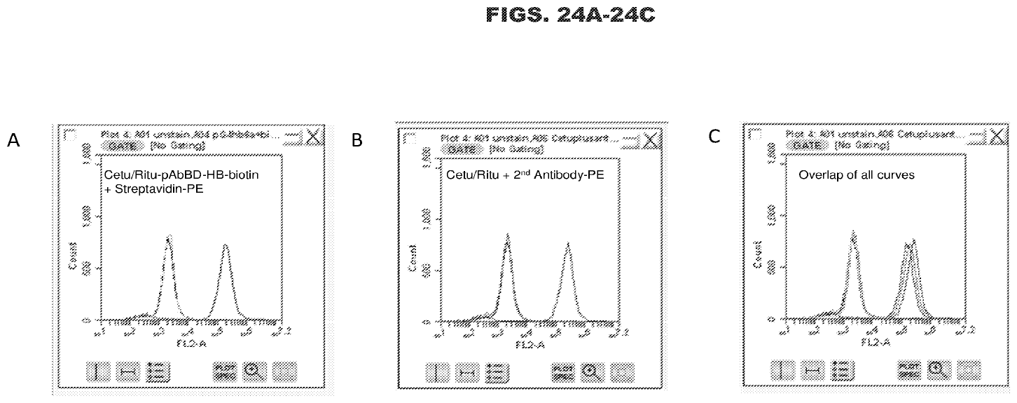

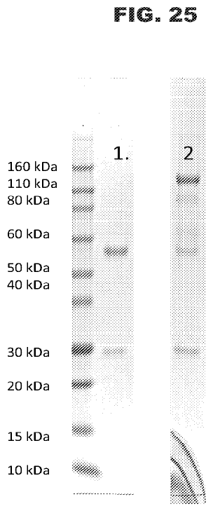

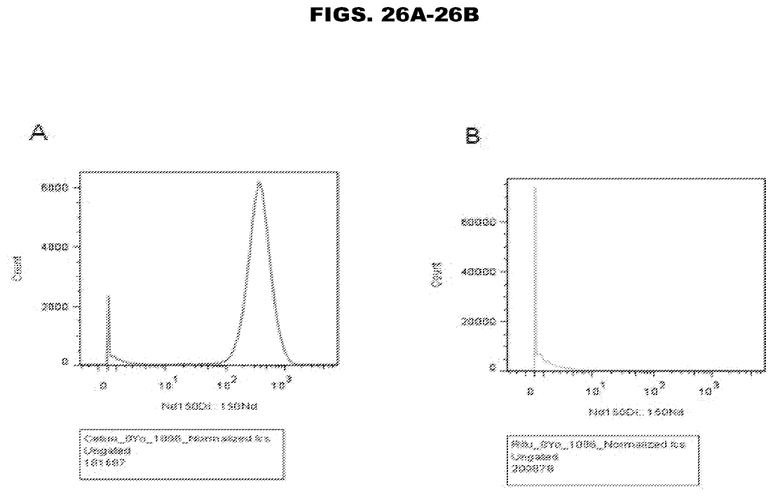

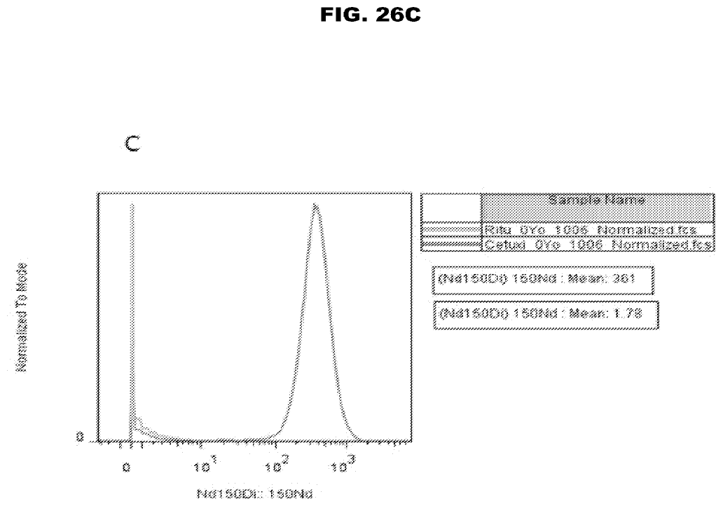

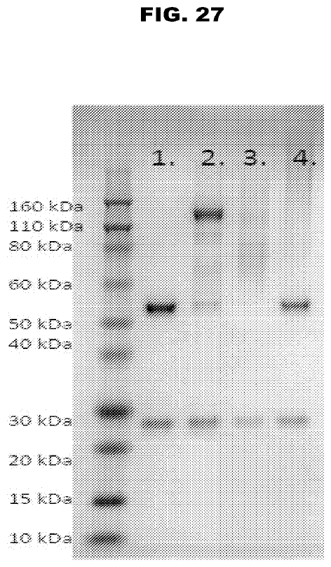

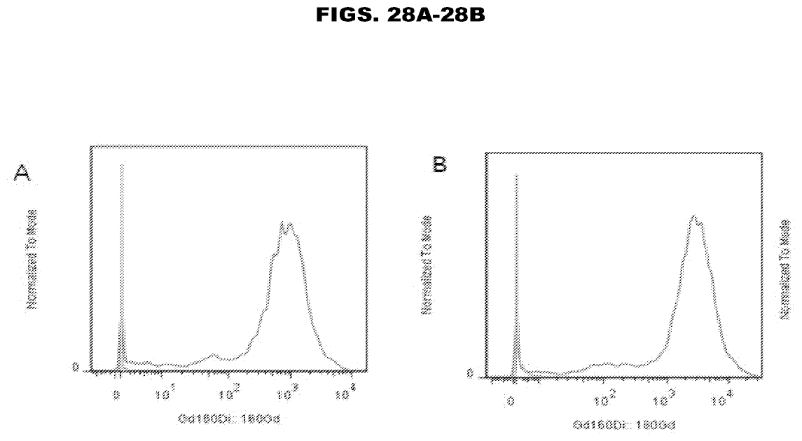

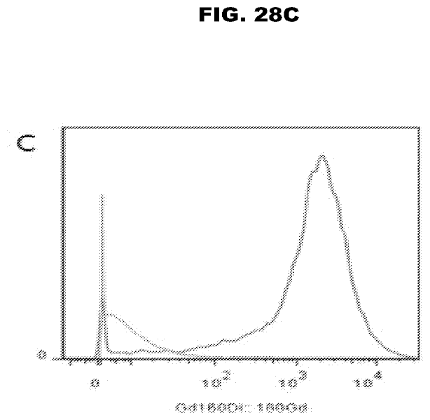

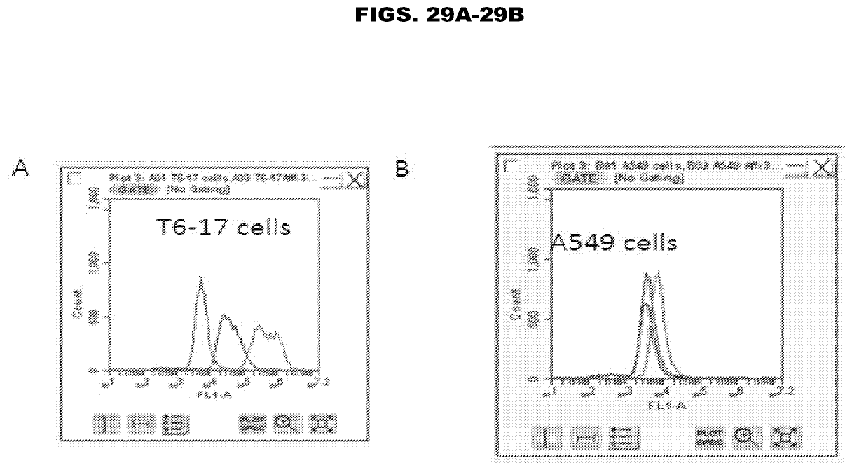

### DETAILED DESCRIPTION OF THE INVENTION

The subject matter here may be understood more readily by reference to the following detailed description which forms a part of this disclosure. It is to be understood that the invention is not limited to the specific products, methods, conditions, or parameters described and/or shown here, and that the terminology used here is for the purpose of describing particular embodiments by way of example only and is not intended to limit the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality,” as used herein, means more than one. When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

In many biomedical applications (e.g. flow cytometry, enzyme-linked immunosorbent assays (ELISA), diagnostic assays, contrast-enhanced imaging, drug delivery, etc.), it is necessary to label antibodies or other targeting ligands with a desired cargo, which can include fluorescent dyes, haptens (e.g. biotin), contrast agents (e.g. gadolinium, radionuclides), therapeutic agents, sensitizers, or other small molecules. Targeting ligands can include proteins, small molecules, aptamers, peptides, etc. Labeling of these targeting ligands can be achieved by a variety of approaches. For example, the antibody or protein can be labeled directly with the cargo, whereby the cargo is covalently attached to nucleophiles such as the side chains of lysines or cysteines that are available for chemical reactions. While this approach is widely adopted due to its simplicity, it has shortcomings, such as a lack of control over which nucleophiles are labeled, potential interference of the cargo with normal protein function, and a limitation in the amount of cargo that can be attached. In some applications, such as labeling of antibodies/proteins with fluorescent dyes, there can also be significant self-quenching if the fluorescent dyes are too close in proximity. To enable more control over the labeling of antibodies or proteins, nucleophiles or unnatural amino acids can be incorporated into the coding sequence to allow for site-specific labeling with cargo. However, many of the same limitations still exist (e.g. low payload). These limitations can sometimes be overcome with the use of secondary antibodies, which can be labeled with the desired cargo and selected to bind the primary antibody. Since multiple secondary antibodies can bind a single primary antibody, the amount of cargo that can be bound to the primary antibody can be quite extensive. However, the use of secondary antibodies is generally limited to in vitro (research or diagnostic) assays. Moreover, binding is non-covalent, which can lead to dissociation of the secondary antibody from the primary antibody under some circumstances. In addition, the use of secondary antibodies can limit multiplexing capabilities in applications such as flow cytometry, since unique primary and secondary antibodies must be carefully paired.

An alternative approach for labeling of antibodies/proteins with cargo involves the use of nanoparticles. Nanoparticles can be loaded/functionalized with high payloads of cargo; however, nanoparticles are generally much bigger than antibodies/proteins, are generally not precisely defined structures, can diffuse very slow due to their large size, can exhibit a high level of non-specific interactions, and for in vivo applications can exhibit poor tissue penetration. Therefore, nanoparticles are not suitable or not desirable for many biomedical applications.

According to one approach provided herein, labeling antibodies/proteins with a high payload of cargo involves the covalent conjugation or fusion of a protein scaffold that possesses or has been engineered to include nucleophiles or unnatural amino acids at precisely defined positions. The desired cargo is attached to the engineered (or naturally nuceleophilic) sites on the protein scaffold. This approach allows for precise control over the location of the cargo on the protein scaffold, which can be used to achieve optimal functionality. For example, fluorescent dyes can be precisely positioned so as to minimize fluorescent self-quenching. In addition to altering the number of engineered sites on the protein scaffold, the size of the protein scaffold can also be adjusted to dictate the number of cargo that can be attached.

The protein scaffold can be attached to antibodies/proteins or other targeting ligands via several different approaches. The simplest approach involves genetic fusion, whereby the coding sequence for the protein scaffold is cloned in frame with the targeting ligand. A second approach involves the introduction of a reactive chemical moiety onto the protein scaffold to enable its attachment to the desired antibody/protein. Common reactive groups include NHS esters, maleimide, free thiols (e.g. cysteine), click chemistry groups (azide, alkyne, constrained alkyne), etc. A third approach involves the fusion of the protein scaffold to a domain that can bind the protein/antibody. This domain can further include a photoreactive group (or other chemically-reactive group) that enables the covalent attachment of the protein scaffold to the protein/antibody.

While any protein could presumably serve as a protein scaffold, some structures provide more favorable options, such as helical bundles and beta barrels, due to their well-defined and rigid structure. These structures also do not naturally contain any cysteines, which allows for cysteines to be engineered into these structures and used as nucleophiles for site-specific labeling. Unnatural amino acids, lysines, or other nucleophiles can also be incorporated into the protein scaffold and used as attachment sites for cargo, through engineering of surface exposed amino acids.

In one aspect, provided herein are protein scaffolds and compositions thereof comprising: (i) a helical bundle having a plurality of chemical handles and (ii) cargo, wherein the chemical handles have been labeled with the cargo. In one embodiment, the chemical handles can be a lysine, a cysteine or a combination thereof. In one embodiment, the cargo includes, but is not limited to, fluorescent dyes, haptens (e.g. biotin), contrast agents (e.g. gadolinium, radionuclides), chelated metals, therapeutic agents, sensitizers, small molecules, or combinations thereof.

In one aspect, provided herein are protein scaffolds and compositions thereof, comprising: (i) a helical bundle having a plurality of chemical handles at defined locations and (ii) cargo, wherein the chemical handles have been labeled with the cargo. In one embodiment, the chemical handles can be a lysine, a cysteine, an unnatural amino acid or a combination thereof, and wherein the chemical handles have been labeled with cargo. In one embodiment, the cargo includes, but is not limited to, fluorescent dyes, haptens (e.g. biotin), contrast agents (e.g. gadolinium, radionuclides), chelated metals, therapeutic agents, sensitizers, small molecules, or combinations thereof. In some embodiments, the chemical handles have been introduced at high density on the surface of the helical bundle. In some embodiments, the chemical handles have been labeled with fluorescent dyes, and the chemical handles are spaced so as to limit the quenching of the fluorescent dyes. In one embodiment, the fluorescent dye emits a photon. In another embodiment, the fluorescent dye is a photosensitizer, wherein the photosensitizer can generate a reactive oxygen species.

In one aspect, provided herein are protein scaffolds and compositions thereof comprising: a plurality of helical bundles in tandem labeled with cargo, wherein each helical bundle has a plurality of chemical handles, and wherein the chemical handles have been labeled with the cargo. In one embodiment, the chemical handles can be a lysine, a cysteine or a combination thereof. In one embodiment, the cargo includes, but is not limited to, fluorescent dyes, haptens (e.g. biotin), contrast agents (e.g. gadolinium, radionuclides), chelated metals, therapeutic agents, sensitizers, small molecules, or combinations thereof.

In one aspect, provided herein are protein scaffolds and compositions thereof comprising: a helical bundle, wherein the helical bundle has been designed to include a plurality of a first chemical handle, and a single second chemical handle that is distinct from the first, wherein the first chemical handle can be a lysine, a cysteine, an unnatural amino acid or combination thereof, wherein the second chemical handle can be a lysine, a cysteine, an amine, a thiol, an unnatural amino acid, a click-chemistry group, a thiol-reactive moiety, or an amine-reactive moiety, and wherein the first chemical handle is labeled with cargo, and wherein the second chemical handle allows for the attachment of said helical bundle to a protein, nucleic acid, small molecule, particle, or surface. In one embodiment, the cargo includes, but is not limited to, fluorescent dyes, haptens (e.g. biotin), contrast agents (e.g. gadolinium, radionuclides), chelated metals, therapeutic agents, sensitizers, small molecules, or combinations thereof.

In one aspect, provided herein are protein scaffolds and compositions thereof comprising: a helical bundle that has been operably linked to a moiety selected from a protein, a nucleic acid, a polymer, a lipid, a small molecule or a combination thereof, and wherein the helical bundle has been labeled with a plurality of cargo. In some embodiments, the moiety is a targeting ligand. In one embodiment, the moiety is an antibody-binding domain (AbBD). In some embodiments, the antibody binding domain is operably linked to a photoreactive amino acid group, for example, benzoylphenylalanine (BPA) resulting in a photoreactive antibody binding domain (pAbBD). In some embodiments, the AbBD or pAbBD is operably linked to an antibody. In one embodiment, the cargo includes, but is not limited to, fluorescent dyes, haptens (e.g. biotin), contrast agents (e.g. gadolinium, radionuclides), chelated metals, therapeutic agents, sensitizers, small molecules, or combinations thereof.

In one aspect, provided herein are methods for imaging and/or detecting cells (e.g., tumor cells) in vitro, comprising: (a) contacting the cells with a protein scaffold described herein that is operably linked to a targeting ligand, wherein the targeting ligand binds to a component of the cells; and imaging and/or detecting the cells by visualizing and/or detecting the cargo of the protein scaffold.

In one aspect, provided herein are methods for imaging cells or tissue (e.g., tumor cells or tumor tissue) in a subject, comprising: (a) administering to the subject a protein scaffold described herein that is operably linked to a targeting ligand, wherein the targeting ligand binds to a component of the cells or tissue; and visualizing the cells or tissue by detecting the cargo of the protein scaffold.

In one aspect, provided herein are methods for intraoperative optical image-guided surgery of a tumor in a subject, comprising: (a) administering to the subject a protein scaffold described herein that is operably linked to a targeting ligand, wherein the targeting ligand binds to a surface of cancers cells of the tumor; visualizing the tumor and delineating intraoperative margins thereof during the surgery by detecting the cargo of the protein scaffold; and (c) resecting the tumor at or near the delineated intraoperative margins thereof.

In another aspect, provided herein are vectors encoding the protein scaffolds and other protein compositions described herein. In some embodiments, the vector is an expression vector. In still another aspect, provided herein is a cell for recombinantly expressing the protein scaffolds and other protein compositions described herein, where the cell is a bacterial cell, yeast cell, insect cell, or mammalian cell. In some embodiments, the cell is transformed with an expression vector described herein.

In some embodiments, the helical bundle is a 4-helical bundle (4HB). In some embodiments, the helical bundle is a 6-helical bundle. In some embodiments, the helical bundle is an 8-helical bundle. In some embodiments, the helical bundle is one that ranges from a 2-helical bundle to an 8-helical bundle.

In some embodiments, the helical bundle has 3 to 50 chemical handles at the defined locations. In some embodiments, the helical bundle has 3 to 10 chemical handles at the defined locations. In some embodiments, the helical bundle has 3 to 6 chemical handles at the defined locations. In some embodiments, the helical bundle has 6 to 50 chemical handles at the defined locations. In some embodiments, the helical bundle has 6 to 10 chemical handles at the defined locations. In some embodiments, the helical bundle has 10 to 50 chemical handles at the defined locations.

In some embodiments, the helical bundle is labeled with 3 to 50 NIR fluorescent dyes with little to no self-quenching. In some embodiments, the helical bundle is labeled with 3 to 10 NIR fluorescent dyes with little to no self-quenching. In some embodiments, the helical bundle is labeled with 3 to 6 NIR fluorescent dyes with little to no self-quenching. In some embodiments, the helical bundle is labeled with 6 to 50 NIR fluorescent dyes with little to no self-quenching. In some embodiments, the helical bundle is labeled with 6 to 10 NIR fluorescent dyes with little to no self-quenching. In some embodiments, the helical bundle is labeled with 10 to 50 NIR fluorescent dyes with little to no self-quenching.

In some embodiments, an antibody binding domain (AbBD) comprises Protein A, Protein G, Protein L, CD4, or a subdomain thereof. In some embodiments, said subdomain is an engineered subdomain, such as to include a non-natural amino acid, a photoreactive group, or a crosslinker. In some embodiments, said antibody-binding domain (AbBD) is operably linked to a photoreactive amino acid and is operably linked to an antibody or a fragment thereof. In one embodiment, said antibody-binding domain (AbBD) is operably linked to an immunoglobulin Fc region, such as an IgG. In one embodiment, said photoreactive amino acid is a UV-active non-natural amino acid or benzoylphenylalaine (BPA). In some embodiments, said antibody-binding domain is a domain of Protein G, Protein A, Protein L, or CD4 or is hyperthermophilic variant of the B1 domain of protein G (HTB1). In some embodiments, BPA is incorporated into a protein Z comprising SEQ ID NO: 31, such as to replace F5, F13, L17, N23, Q32, or K35 of SEQ ID NO: 31. In some embodiments, BPA is incorporated into a protein G domain comprising SEQ ID NO: 32, such as to replace A24 or K28 of SEQ ID NO: 32. Examples of antibody-binding domains (AbBDs) are described in US2016/0041157, US2018/0344871, and US2020/0277403, each of which is incorporated by reference herein in its entirety.

In one embodiment, a variety of radioactive isotopes are available as cargo for the production of protein scaffolds and other proteins and can be of use in the methods and compositions provided herein. Examples include, but are not limited to, At²¹¹, Cu⁶⁴, I¹³¹, I¹²⁵, Y⁹⁰, Re¹⁸⁶, Re¹⁸⁸, Sm¹²⁵, Bi²¹², P³², Zr⁸⁹ and radioactive isotopes of Lu.

The term “Protein Z,” as used herein, refers to the Z domain based on B domain of Staphylococcal aureus Protein A. The amino acid sequence of wild-type Protein Z is: VDNKFNKEQQNAFYEILHLPNLNEEQRNAFIQSLKDDPSQSANLLAEAKKLNDAQAPKMRM (SEQ ID NO: 31). Photoreactive Protein Z includes those where an amino acid in protein Z has been replaced with benzoylphenylalanine (BPA), such as F13BPA and F5BPA (see underlined amino acids in bold in SEQ ID NO: 31). Examples of other BPA-containing mutants of Protein Z include, for example, but are not limited to, Q32BPA, K35BPA, N28BPA, N23BPA, and L17BPA. Examples of Protein Z variants or mutants include, F51, such as F51 K35BPA. The Protein Z amino acid sequence may also include homologous, variant, and fragment sequences having Z domain function. In some embodiments, the Protein Z amino acid sequence may include an amino acid sequence which is 60. 65, 70, 75, 80, 85, 90, 95, or 99% identity to the sequence set forth in SEQ ID NO: 25.

The term “Protein G,” as used herein, refers to a B1 domain based of Streptococcal Protein G. Preferably, the Protein G is a hypothermophilic variant of a B1 domain based of Streptococcal Protein G. The amino acid sequence of Protein G preferably is: MTFKLIINGKTLKGEITIEAVDAAEAEKIFKQYANDYGIDGEWTYDDATKTFTVTE (SEQ ID NO: 32) as described in WO2016/183387, published Nov. 17, 2016, which is incorporated herein by reference in its entirety. As further described in WO2016/183387, nine Protein G variants were successfully designed and expressed, each having an Fc-facing amino acid substituted by BPA: V21, A24, K28, I29, K31, Q32, D40, E42, W42 (see underlined amino acids in bold in SEQ ID NO: 32). Two variants, A24BPA and K28BPA, allowed ˜100% of all human IgG subtypes to be labeled. The Protein G amino acid sequence may also include homologous, variant, and fragment sequences having B1 domain function. In some embodiments, the Protein G amino acid sequence may include an amino acid sequence which is 60, 65, 70, 75, 80, 85, 90, 95, or 99% identity to the sequence set forth in SEQ ID NO: 32.

As used herein, the term “antibody” encompasses the structure that constitutes the natural biological form of an antibody. In most mammals, including humans, and mice, this form is a tetramer and consists of two identical pairs of two immunoglobulin chains, each pair having one light and one heavy chain, each light chain comprising immunoglobulin domains VL and CL, and each heavy chain comprising immunoglobulin domains VH, Cγ1, Cγ2, and Cγ3. In each pair, the light and heavy chain variable regions (VL and VH) are together responsible for binding to an antigen, and the constant regions (CL, Cγ1, Cγ2, and Cγ3, particularly Cγ2, and Cγ3) are responsible for antibody effector functions. In some mammals, for example in camels and llamas, full-length antibodies may consist of only two heavy chains, each heavy chain comprising immunoglobulin domains VH, Cγ2, and Cγ3. By “immunoglobulin (Ig)” herein is meant a protein consisting of one or more polypeptides substantially encoded by immunoglobulin genes. Immunoglobulins include but are not limited to antibodies. Immunoglobulins may have a number of structural forms, including but not limited to full-length antibodies, antibody fragments, and individual immunoglobulin domains including but not limited to VH, Cγ1, Cγ2, Cγ3, VL, and CL.

Depending on the amino acid sequence of the constant domain of their heavy chains, intact antibodies can be assigned to different “classes”. There are five-major classes (isotypes) of intact antibodies: IgA, IgD, IgE, IgG, and IgM, and several of these may be further divided into “subclasses”, e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known to one skilled in the art.

The term “antibody” or “antigen-binding fragment” respectively refer to intact molecules as well as functional fragments thereof, such as Fab, a scFv-Fc bivalent molecule, F(ab')2, and Fv that are capable of specifically interacting with a desired target. Antigen-binding fragments include:

(1) Fab, the fragment which contains a monovalent antigen-binding fragment of an antibody molecule, which can be produced by digestion of whole antibody with the enzyme papain to yield an intact light chain and a portion of one heavy chain;

(2) Fab', the fragment of an antibody molecule that can be obtained by treating whole antibody with pepsin, followed by reduction, to yield an intact light chain and a portion of the heavy chain; two Fab' fragments are obtained per antibody molecule;

(3) (Fab')2, the fragment of the antibody that can be obtained by treating whole antibody with the enzyme pepsin without subsequent reduction; F(ab')2 is a dimer of two Fab' fragments held together by two disulfide bonds;

(4) Fv, a genetically engineered fragment containing the variable region of the light chain and the variable region of the heavy chain expressed as two chains; and

(5) Single chain antibody (“SCA”), a genetically engineered molecule containing the variable region of the light chain and the variable region of the heavy chain, linked by a suitable polypeptide linker as a genetically fused single chain molecule.

(6) scFv-Fc, is produced by fusing single-chain Fv (scFv) with a hinge region from an immunoglobulin (Ig) such as an IgG, and Fc regions.

In some embodiments, an antibody provided herein is a monoclonal antibody. In some embodiments, the antigen-binding fragment provided herein is a single chain Fv (scFv), a diabody, a tandem scFv, a scFv-Fc bivalent molecule, an Fab, Fab', Fv, F(ab')2 or an antigen binding scaffold (e.g., affibody, monobody, anticalin, DARPin, Knottin, etc.). “Affibodies” are small proteins engineered to bind to a large number of target proteins or peptides with high affinity, often imitating monoclonal antibodies, and are antibody mimetics.

As used herein, the terms “bivalent molecule” or “BV” refer to a molecule capable of binding to two separate targets at the same time. The bivalent molecule is not limited to having two and only two binding domains and can be a polyvalent molecule or a molecule comprised of linked monovalent molecules. The binding domains of the bivalent molecule can selectively recognize the same epitope or different epitopes located on the same target or located on a target that originates from different species. The binding domains can be linked in any of a number of ways including, but not limited to, disulfide bonds, peptide bridging, amide bonds, and other natural or synthetic linkages known in the art.

As used herein, the terms “binds” or “binding” or grammatical equivalents, refer to compositions having affinity for each other. “Specific binding” is where the binding is selective between two molecules. A particular example of specific binding is that which occurs between an antibody and an antigen. Typically, specific binding can be distinguished from non-specific when the dissociation constant (K_{D}) is less than about 1×10⁻⁵ M or less than about 1×10⁻⁶ M or 1×10⁻⁷ M. Specific binding can be detected, for example, by ELISA, immunoprecipitation, coprecipitation, with or without chemical crosslinking, two-hybrid assays and the like. Appropriate controls can be used to distinguish between “specific” and “non-specific” binding.

TABLE 1

| SEQ | | |
|---|---|---|
| ID | | |
| NO: | Name | Amino Acid Sequence |
| 1 | 4HBWT | DNIEEVKKMLEKMIKEVKKMLENGEDSEKILKKVKEMIEKILKKVKEDGQDPKMIEEVK |
| | | KMLEKMIKEVKKMLENGEDSEKILKKVKEMAEKILKKVKELGVGSGWLE |
| 2 | 4HB4a | DNIEEVCKMLEKMIKEVKKMLENGEDCEKILKKVKEMIEKILKKVKEDGQDPCMIEEV |
| | | KKMLEKMIKEVKKMLENGEDCEKILKKVKEMAEKILKKVKELGVGSGWLE |
| 3 | 4HB4b | DNIEEVCKMLEKMIKEVKKMLENGEDSEKILKKVCEMIEKILKKVKEDGQDPKMIEEVK |
| | | KMLEKMIKEVKCMLENGEDSEKILKKVCEMAEKILKKVKELGVGSGWLE |
| 4 | 4HB6a | DNIEEVCKMLEKMIKEVKCMLENGEDSEKILKKVCEMIEKILKKVKEDGQDPKMIEEV |
| | | CKMLEKMIKEVKCMLENGEDSEKILKKVCEMAEKILKKVKELGVGSGWLE |
| 5 | 4HB6b | DNIEEVCKMLEKMIKEVKCMLENGEDSECILKKVKEMIECILKKVKEDGQDPKMIEEV |
| | | KKMLECMIKEVKKMLENGEDSEKILKKVCEMAEKILKKVKELGVGSGWLE |
| 6 | 4HB8a | DNIEEVCKMLEKMIKEVCKMLENGEDSECILKKVKEMIECILKKVKEDGQDPKMIEEV |
| | | CKMLEKMIKEVKCMLENGEDSEKILKCVKEMAEKILKCVKELGVGSGWLE |
| 7 | 4HB8b | DNIEEVCKMLEKMICEVKKMLENGEDSEKILCKVKEMIECILKKVKEDGQDPKMIEEV |
| | | KCMLEKMIKEVKCMLENGEDSECILKKVKEMAECILKKVKELGVGSGWLE |
| 8 | 4HB30c | DNIEEVCCELECMICEVCCELENGEDSECILCCVCEEIECILCCVCEDGQDPCMIEEVCC |
| | | MLECMICEVCCMLENGEDSECILCCVCEMAECILCCVCELGVGSGWLC |
| 9 | 4hblongWT | DNEEVKKMLEKMIEEIKKMLEKAIKKVKEMLEKMIKEIKKMLENGEDSEKILKKAKEM |
| | | AEKILKMVIELAEKILKKAKEMAEKILKKVKELGVDNEEVKKMLEKMIEEIKKMLEKAIK |
| | | KVKEMLEKMIKEIKKMLENGEDSEKILKKAKEMAEKILKMVIELAEKILKKAKEMAEKIL |
| | | KKVKELGVGGW |
| 10 | 4hblong8c | DNECVKKMLEKMIEEIKKMLEKAIKKVKCMLEKMIKEIKKMLENGEDSECILKKAKEM |
| | | AEKILKMVIELAEKILCKAKEMAEKILKKVKELGVDNEEVKCMLEKMIEEIKKMLEKAIK |
| | | KVKCMLEKMIKEIKKMLENGEDSEKILCKAKEMAEKILKMVIELAEKILCKAKEMAEKIL |
| | | KKVKELGVGGW |
| 11 | 4hblong8c | DNECVKKMLEKMIEEIKKMLECAIKKVKEMLEKMIKEIKCMLENGEDSEKILKKAKEM |
| | | AECILKMVIELAEKILKKAKCMAEKILKKVKELGVDNECVKKMLEKMIEEIKKMLECAIK |
| | | KVKEMLEKMIKEIKCMLENGEDSEKILKKACEMAEKILKMVIELAEKILKKACEMAEKIL |
| | | KKVKELGVGGW |
| 12 | 4HB6a3ME | DNIEEVCKELEKMIKEVKCELENGEDSEKILKKVCEEIEKILKKVKEDGQDPKMIEEVCK |
| | | MLEKMIKEVKCMLENGEDSEKILKKVCEMAEKILKKVKELGVGSGWLE |
| 13 | HB1P68WT | YGKLNDLLEDLQEVLKNLHKNWHGGKDNLHDVDNHLQNVIEDIHDFMQGGGSGGK |
| | | LQEMMKEFQQVLDELNNHLQGGKHTVHHIEQNIKEIFHHLEELVHR |
| 14 | HB1P686c | YGKLNCLLEDLQEVLCNLHKNWHGGKDNLHDVDNHLQCVIEDIHDFMQGGGSGGC |
| | | LQEMMKEFQQVLDCLNNHLQGGKHTVHHIEQNICEIFHHLEELVHR |
| 15 | HB5CWMWT | DPEDELKRVEKLVKEAEELLRQAKEKGSEEDLEKALRTAEEAAREAKKVLEQAEKEGDP |
| | | EVALRAVELVVRVAELLLRIAKESGSEEALERALRVAEEAARLAKRVLELAEKQGDPEVA |
| | | LRAVELVVRVAELLLRIAKESGSEEALERALRVAEEAARLAKRVLELAEKQGDPEVARR |
| | | AVELVKRVAELLERIARESGSEEAKERAERVREEARELQERVKELREREGGWLE |
| 16 | HB5CWM3c | DPEDELKRVEKLVKEAEELLRQAKEKGSEEDLECALRTAEEAAREAKKVLEQAEKEGDP |
| | | EVALRAVELVVRVAELLLRIAKESGSEEALERALRVAEEAARLAKRVLELAECQGDPEVA |
| | | LRAVELVVRVAELLLRIAKESGSEEALERALRVAEEAARLAKRVLELAEKQGDPEVARR |
| | | AVELVKRVAELLERIARESGSEEACERAERVREEARELQERVKELREREGGWLE |
| 17 | HB5CWM4c | DPEDELCRVEKLVKEAEELLRQAKEKGSEEDLEKALRTAEEAAREAKKVLEQAEKEGDP |
| | | EVALRAVELVVRVAELLLRIACESGSEEALERALRVAEEAARLAKRVLELAECQGDPEV |
| | | ALRAVELVVRVAELLLRIAKESGSEEALERALRVAEEAARLAKRVLELAEKQGDPEVAR |
| | | RAVELVKRVAELLERIARESGSEEACERAERVREEARELQERVCELREREGGWLE |
| 18 | HB5CWM5c | DPEDELKRVECLVKEAEELLRQAKEKGSEEDLECALRTAEEAAREACKVLEQAEKEGDP |
| | | EVALRAVELVVRVAELLLRIACESGSEEALERALRVAEEAARLAKRVLELAECQGDPEVA |
| | | LRAVELVVCVAELLLRIAKESGSEEALERALCVAEEAARLACRVLELAEKQGDPEVARRA |
| | | VELVCRVAELLERIARESGSEEACERAERVREEARELQERVCELREREGGWLE |
| 19 | HB5CWM11c | DPEDELKRVECLVKEAEELLRQAKEKGSEEDLECALRTAEEAAREACKVLEQAEKEGDP |
| | | EVALRAVELVVRVAELLLRIACESGSEEALERALRVAEEAARLAKRVLELAECQGDPEV |
| | | ALRAVELVVCVAELLLRIAKESGSEEALERALCVAEEAARLACRVLELAEKQGDPEVAR |
| | | RAVELVCRVAELLERIARESGSEEACERAERVREEARELQERVCELREREGGWLE |
| 20 | HB5CWM20c | DPEDELCRVEKLVCEAEELLRQACEKGSEEDLECALRTAEEAAREACKVLEQAECEGDP |
| | | EVALRAVELVVRVAELLLRIACESGSEEALECALRVAEEAARLACRVLELAECQGDPEV |
| | | ALRAVELVVCVAELLLRIACESGSEEALERALCVAEEAARLACRVLELAECQGDPEVAR |
| | | RAVELVCRVAELLECIARESGSEEACERAERVCEEARELQERVCELREREGGWLE |
| 21 | (HB5CWM20c)2 | DPEDELCRVEKLVCEAEELLRQACEKGSEEDLECALRTAEEAAREACKVLEQAECEGDP |
| | | EVALRAVELVVRVAELLLRIACESGSEEALECALRVAEEAARLACRVLELAECQGDPEV |
| | | ALRAVELVVCVAELLLRIACESGSEEALERALCVAEEAARLACRVLELAECQGDPEVAR |
| | | RAVELVCRVAELLECIARESGSEEACERAERVCEEARELQERVCELREREGGWLEGGS |
| | | GGSDPEDELCRVEKLVCEAEELLRQACEKGSEEDLECALRTAEEAAREACKVLEQAECE |
| | | GDPEVALRAVELVVRVAELLLRIACESGSEEALECALRVAEEAARLACRVLELAECQGD |
| | | PEVALRAVELVVCVAELLLRIACESGSEEALERALCVAEEAARLACRVLELAECQGDPEV |
| | | ARRAVELVCRVAELLECIARESGSEEACERAERVCEEARELQERVCELREREGGWLEG |
| | | GGSGGSLPETGGG |
| 22 | (HB5CWM20c)3 | QGDPEVALRAVELVVCVAELLLRIACESGSEEALERALCVAEEAARLACRVLELAECQG |
| | | DPEDELCRVEKLVCEAEELLRQACEKGSEEDLECALRTAEEAAREACKVLEQAECEGDP |
| | | EVALRAVELVVRVAELLLRIACESGSEEALECALRVAEEAARLACRVLELAECQGDPEV |
| | | ALRAVELVVCVAELLLRIACESGSEEALERALCVAEEAARLACRVLELAECQGDPEVAR |
| | | RAVELVCRVAELLECIARESGSEEACERAERVCEEARELQERVCELREREGGWLEGGS |
| | | GGSGGSDPEDELCRVEKLVCEAEELLRQACEKGSEEDLECALRTAEEAAREACKVLEQ |
| | | AECEGDPEVALRAVELVVRVAELLLRIACESGSEEALECALRVAEEAARLACRVLELAEC |
| | | DPEVARRAVELVCRVAELLECIARESGSEEACERAERVCEEARELQERVCELREREGG |
| | | WLEGGSGGSDPEDELCRVEKLVCEAEELLRQACEKGSEEDLECALRTAEEAAREACKV |
| | | LEQAECEGDPEVALRAVELVVRVAELLLRIACESGSEEALECALRVAEEAARLACRVLEL |
| | | AECQGDPEVALRAVELVVCVAELLLRIACESGSEEALERALCVAEEAARLACRVLELAEC |
| | | QGDPEVARRAVELVCRVAELLECIARESGSEEACERAERVCEEARELQERVCELREREG |
| | | GWLE |
| 23 | HB5CWM46c | DPEDELCCVECLVCEAEELLRQACECGSEEDLECALCTAEEAACEACCVLEQAECEGDP |
| | | EVALCAVELVVCVAELLLCIACESGSEEALECALCVAEEAACLACCVLELAECQGDPEVA |
| | | LCAVELVVCVAELLLCIACESGSEEALECALCVAEEAACLACCVLELAECQGDPEVACCA |
| | | VELVCCVAELLECIACESGSEEACECAECVCEEACELQECVCELCECEGGWLE |
| 24 | (HB5CWM46c)2 | DPEDELCCVECLVCEAEELLRQACECGSEEDLECALCTAEEAACEACCVLEQAECEGDP |
| | | EVALCAVELVVCVAELLLCIACESGSEEALECALCVAEEAACLACCVLELAECQGDPEVA |
| | | LCAVELVVCVAELLLCIACESGSEEALECALCVAEEAACLACCVLELAECQGDPEVACCA |
| | | VELVCCVAELLECIACESGSEEACECAECVCEEACELQECVCELCECEGGWLEGGSGGS |
| | | DPEDELCCVECLVCEAEELLRQACECGSEEDLECALCTAEEAACEACCVLEQAECEGDP |
| | | EVALCAVELVVCVAELLLCIACESGSEEALECALCVAEEAACLACCVLELAECQGDPEVA |
| | | LCAVELVVCVAELLLCIACESGSEEALECALCVAEEAACLACCVLELAECQGDPEVACCA |
| | | VELVCCVAELLECIACESGSEEACECAECVCEEACELQECVCELCECEGGWLE |
| 25 | 3HBWT | NEDDMKKLYKQMVQELEKARDRMEKLYKEMVELIQKAIELMRKIFQEVKQEVEKAIE |
| | | EMKKLYDEAKKKIEQMIQQIKQGGDKQKMEELLKRAKEEMKKVKDKMEKLLEKLKQI |
| | | MQEAKQKMEKLLKQLKEEMKKMKEKMEKLLKEMKQRMEEVKKKMDGDDELLEKIK |
| | | KNIDDLKKIAEDLIKKAEENIKEAKKIAEQLVKRAKQLIEKAKQVAEELIKKILQLIEKA |
| | | KEIAEKVLKGLE |
| 26 | 3HBM/L | NEDDMKKLYKQMVQELEKARDRMEKLYKEMVELIQKAIELMRKIFQEVKQEVEKAIE |
| | | EMKKLYDEAKKKIEQMIQQIKQGGDKQKMEELLKRAKEEMKKVKDKMEKLLEKLKQI |
| | | MQEAKQKMEKLLKQLKEEMKKMKEKMEKLLKEMKQRMEEVKKKMDGDDELLEKIK |
| | | KNIDDLKKIAEDLIKKAEENIKEAKKIAEQLVKRAKQLIEKAKQVAEELIKKILQLIEKA |
| | | KEIAEKVLKGLE |
| 27 | 3HBM/L3C | NEDDMKKLYKQMVQELCKARDRMEKLYKEMVELIQKAIELMRKIFQEVKQEVEKAIE |
| | | EMKKLYDEAKKKIEQMIQQIKQGGDKQKMEELLKRAKEEMKKVKDKMEKLLEKLKQI |
| | | MQEAKCKMEKLLKQLKEEMKKMKEKMEKLLKEMKQRMEEVKKKMDGDDELLEKIK |
| | | KNIDDLKKIAEDLIKKAEENIKEAKKIAEQLVKRAKQLIEKAKQVAEELIKKILQLICKA |
| | | KEIAEKVLKGLE |
| 28 | 3HBM/L4C | NEDDLKKLYKQLVQELEKARDRLECLYKELVELIQKAIELLRKIFQEVKQEVEKAIEELKK |
| | | LYDEAKKKIECLIQQIKQGGDKQKLEELLKRAKEELKKVKDKLEKLLEKLKQILQEAKQKL |
| | | EKLLKQLKEELKKLKEKLEKLLKELKQRLEEVKKCLDGDDELLEKIKKNIDDLKKIAEDLI |
| | | KKAEENIKEAKKIAEQLVKRAKQLIEKAKQVAEELIKKILCLIEKAKEIAEKVLKGLE |
| 29 | 3HBM/L5C | NEDDLKKLYKQLVQELEKARDRLEKLYKCLVELIQKAIELLRKIFQEVKQEVEKAIEELKK |
| | | LYDEAKKKIEQLIQQICQGGDKQKLEELLKRAKEELKKVKDKLEKLLEKLKQILCEAKQKL |
| | | EKLLKQLKEELKKLKEKLEKLLKELKQRLEEVKKKLDGDDELLCKIKKNIDDLKKIAEDLI |
| | | KKAEENIKEAKKIAEQLVKRAKQLIEKAKQVAECLIKKILQLIEKAKEIAEKVLKGLE |
| 30 | 3HBM/L6C | NEDDMKKLYKQMVQCLEKARDRMEKLYKEMVELIQKAIELMRKIFQEVCQEVEKAIE |
| | | EMKKLYDEAKKKIEQMIQQIKQGGDCQKMEELLKRAKEEMKKVKDKMEKLLEKLKQI |
| | | MQEAKQKMEKLLCQLKEEMKKMKEKMEKLLKEMKQRMEEVKKKMDGDDELLEKIKKN |
| | | IDDLKKIACDLIKKAEENIKEAKKIAEQLVKRAKQLIEKAKQVAECLIKKILQLIEKAKE |
| | | IAEKVLKGLE |

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviations, per practice in the art. Alternatively, when referring to a measurable value such as an amount, a temporal duration, a concentration, and the like, may encompass variations of ±20% or ±10%, more preferably ±5%, even more preferably ±1%, and still more preferably ±0.1% from the specified value, as such variations are appropriate to perform the disclosed methods.

There are many options for operably linking molecules. A variety of linkers may find use in the compositions and methods provided herein. The term “linker,” “linker sequence,” “spacer,” “tethering sequence” or grammatical equivalents thereof refer to a molecule or group of molecules (such as a monomer or polymer) that connects two molecules and often serves to place the two molecules in a preferred configuration. A number of strategies may be used to covalently link molecules together. These include, but are not limited to polypeptide linkages between N- and C-terminus of proteins or protein domains, linkage via disulfide bonds, and linkage via chemical cross-linking reagents. In one aspect of this embodiment, the linker is a peptide bond, generated by recombinant techniques or peptide synthesis. In another embodiment the linker is a cysteine linker. In yet another embodiment, it is a multi-cysteine linker. Choosing a suitable linker for a specific case where two polypeptide chains are to be connected depends on various parameters, including but not limited to the nature of the two polypeptide chains (e.g., whether they naturally oligomerize), the distance between the N- and the C-termini to be connected if known, and/or the stability of the linker towards proteolysis and oxidation. Furthermore, the linker may contain amino acid residues that provide flexibility. Thus, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. Suitable lengths for this purpose include at least one and not more than 30 amino acid residues. In one embodiment, a linker is from about 1 to 30 amino acids in length. In another embodiment, a linker is from about 1 to 15 amino acids in length. In addition, the amino acid residues selected for inclusion in the linker peptide should exhibit properties that do not interfere significantly with the activity of the polypeptide(s). Thus, a linker peptide on the whole should not exhibit a charge that would be inconsistent with the activity of the polypeptide, or interfere with internal folding, or form bonds or other interactions with amino acid residues in one or more of the monomers that would seriously impede the binding of monomer domains. Useful linkers include glycine-serine polymers, glycine-alanine polymers, alanine-serine polymers, and other flexible linkers such as the tether for the shaker potassium channel, and a large variety of other flexible linkers, as will be appreciated by those in the art. Suitable linkers may also be identified by screening databases of known three-dimensional structures for naturally occurring motifs that can bridge the gap between two polypeptide chains. In one embodiment, the linker is not immunogenic when administered in a human subject. Thus linkers may be chosen such that they have low immunogenicity or are thought to have low immunogenicity. Another way of obtaining a suitable linker is by optimizing a simple linker, e.g., (Gly₄Ser)ₙ, through random mutagenesis. Alternatively, once a suitable polypeptide linker is defined, additional linker polypeptides can be created to select amino acids that more optimally interact with the domains being linked. Other types of linkers that may be used in the compositions and methods provided herein include artificial polypeptide linkers and inteins. In another embodiment, disulfide bonds are designed to link the two molecules. In another embodiment, linkers are chemical cross-linking agents. For example, a variety of bifunctional protein coupling agents may be used, including but not limited to N-succinimidyl-3-(2-pyridyldithiol) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). In another embodiment, chemical linkers may enable chelation of an isotope. For example, Carbon-14-labeled 1-isothiocyanatobenzyl-3-methyldiethylene triaminepentaacetic acid (MX-DTPA) is an exemplary chelating agent for conjugation of radionucleotide to the antibody. The linker may be cleavable, facilitating release of the cytotoxic drug in the cell. For example, an acid-labile linker, peptidase-sensitive linker, dimethyl linker or disulfide-containing linker (Chari et al., 1992, Cancer Research 52: 127-131) may be used. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers, that is may find use to link the components of the compositions provided herein.

The term “subject” refers to a mammal including a human in need of therapy for, or susceptible to, a condition or its sequelae. The subject may include dogs, cats, pigs, cows, sheep, goats, horses, rats, and mice and humans. The term “subject” does not exclude an individual that is normal in all respects.

Any patent. patent application publication, or scientific publication, cited herein, is incorporated by reference herein in its entirety.

The following examples are presented in order to more fully illustrate preferred embodiments of the invention. They should in no way be construed, however, as limiting the broad scope of the invention.

### EXAMPLES

### Example 1

### a Targeted, Superbright, Protein-Based Scaffold for Near Infrared Fluorescent Image-Guided Surgery

The majority of women that are diagnosed with breast cancer undergo breast-conserving surgery. Unfortunately, for >20% of these women, it is post-surgically determined that the margins are not tumor free. One potential solution is the use of fluorescent image-guided surgery to improve intraoperative margin assessment. It was found that the near-infrared (NIR) fluorescent dye, indocyanine green (ICG), can be used to help differentiate breast tumor tissue from normal tissue in clinical investigations; however, a low specificity and redistribution of ICG during tumor resection led to false-positives. The use of fluorescently-labeled targeting ligands provide an opportunity to overcome these shortcomings; however, the sensitivity of targeted agents is quite poor, since they can only be labeled with 1 to 3 dyes before self-quenching negatively impacts the fluorescent intensity. Therefore, a protein-based scaffold was developed that can be labeled with up to 10 NIR dyes with little to no self-quenching. The overall goal of this Example is to further optimize the design of these protein scaffolds, create a Her2/neu-targeted variant for breast cancer detection, and test the ability of these imaging agents to accurately identify breast tumor margins. The fluorescent platform developed in this proposal is expected to be applicable for image-guided surgery for a wide range of cancer-types, and have the potential for fast clinical translation.

### BACKGROUND

Breast Cancer: The majority of women that are diagnosed with early stage breast cancer undergo breast-conserving surgery (BCS). Unfortunately, for >20% of these women, it is post-surgically determined that the margins are not tumor free and must be subjected to additional surgery to avoid local recurrence. Therefore, there is an unmet need for improved methods to better identify tumor margins during the initial operation.

Intraoperative Imaging: Current methods of intraoperative margin assessment include frozen section, imprint cytology, intraoperative ultrasound, wire localization, radio-guided localization, and two-view specimen mammography. These methods are labor intensive, time consuming, may lead to poor cosmesis, and in some cases are limited in their ability to assess the entire margin. It is postulated that real-time optical image-guided surgery may be a better option for intraoperative margin assessment.

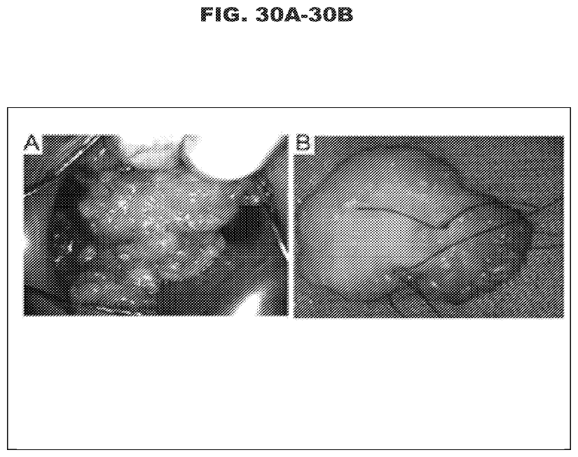

Indocyanine green (ICG) is a near-infrared (NIR) fluorescent dye that is used for real-time image guided surgery. ICG can diffuse into tumors via enhanced permeability and retention. ICG was used in a pre-clinical and clinical investigation of breast cancer. In the clinical trial, the fluorescence from the breast tumors was readily distinguished from normal tissue (FIG. 30); however, a low specificity and redistribution of ICG during tumor resection led to false-positives.

The use of fluorescently-labeled targeting ligands provide an opportunity to improve specificity compared with ICG and prevent the re-distribution and movement of the contrast agent during surgery. For example, folate-targeted dyes were shown to be able to correctly identify 46 out of 50 biopsy-proven human lung adeno-carcinomas; however, the sensitivity of this agent was poor—only 7 out of 50 tumors were seen in vivo. This is partly because fluorescently labeled targeting ligands can only be labeled with 1 to 3 dyes, before self-quenching negatively impacts the fluorescent intensity. Targeted fluorescent nanoparticles offer one possible solution, but nanoparticles suffer from poor tissue penetration with limited ability to reach tumor cells beyond the endothelial wall. The patchy variations in endothelial permeability that exist throughout a tumor therefore result in inconsistent and unpredictable dissemination. Thus, new imaging agents are still needed that can exhibit rapid intratumoral penetration, high contrast and clear delineation of the entire tumor margin.

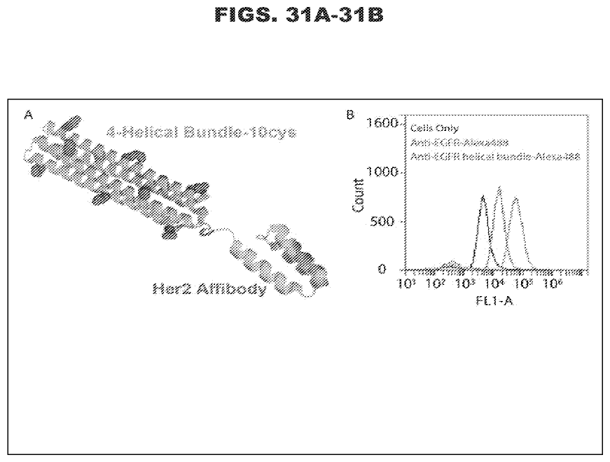

In this Example, a 4-helical bundle (4HB) is used as a compact protein scaffold that can be labeled with up to 10 NIR fluorescent dyes at precisely defined locations to avoid self-quenching (FIG. 31A). Dye-labeled 4HB s can serve as a universal, ‘superbright’ fluorescent platform that can be conjugated or fused to any targeting ligand for image-guided surgery. In this Example, the 4HB is fused to a Her2/neu-targeted affibody. Due to the high payload of unquenched NIR dyes on the 4HB, this imaging agent is expected to be at least 5-times brighter than targeting ligands that have been directly labeled with NIR dyes using conventional approaches. Moreover, the 4HB is expected to be smaller and brighter than fluorescently labeled linear (e.g. poly-lysine, dextran) and branched polymers (dendrons), because the 4HB is a precisely defined molecular entity that can engineered with NIR dyes positioned at pre-defined locations to avoid self-quenching, as opposed to polydisperse dye-labeled polymer assemblies. The 4HB is also expected to exhibit better tissue penetration and distribution, compared with fluorescent nanoparticles, due to its small size.

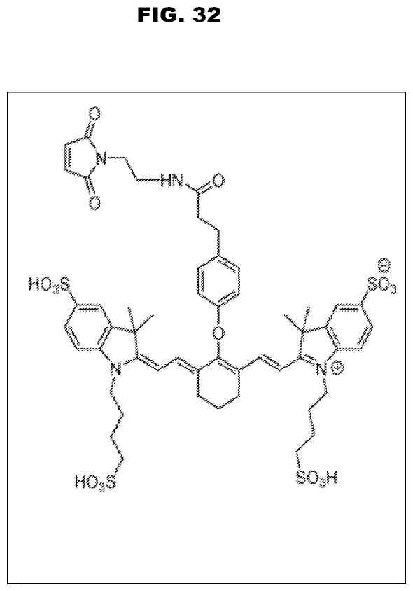

The 4HB to be utilized has a molecular weight of 23 kDa, which is small enough for renal filtration, even when produced as a fusion protein with the Her2/neu-targeted affibody (6 kDa). If a longer circulation time is desired, the construct could easily be modified with PEG. The 4HB is genetically engineered with cysteines at defined locations, to allow for site-specific labeling with the thiol-reactive NIR dye S0456-maleimide (FIG. 32). The dyes are strategically positioned to prevent self-quenching. S0456 is utilized clinically, like ICG, and possess similar optical properties, but is more photostable.

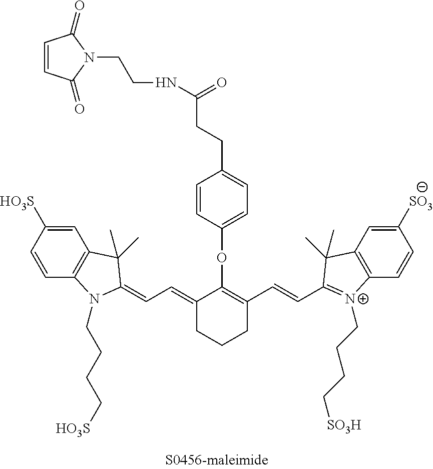

Since 4HBs are found throughout nature, including within many mammalian proteins (e.g. ferritin, human growth hormone, cytokines), these agents are expected to elicit little to no immunogenic response.

Prepare and Characterize Superbright, S0456-Labeled Helical Bundles 4HBs have been recombinantly expressed and purified as fusion proteins with photoreactive antibody binding domains (pAbBDs). The 4HB was engineered with 10 strategically placed cysteine residues that were labeled the green fluorescent dye, Alexa488. Anti-EGFR antibodies were conjugated with these helical bundles, incubated with EGFR-positive cells, and cell labeling was assessed via fluorescence microscopy and flow cytometry. Cells incubated with the EGFR-targeted-4HBs exhibited a >5-times higher mean fluorescence than cells incubated with an equivalent dose of anti-EGFR antibodies that were directly labeled with Alexa488. Direct labeling with Alexa488 was performed according to the manufacturer's instructions (reaction ratio of 20:1) (FIG. 31B). In this Example, the 4HB is fused to a Her2/neu-targeted affibody and the conjugate is labeled with S0456-maleimide. Note: Neither the 4HB nor the affibody include cysteines in their native amino acid sequence. S0456-maleimide is synthesized as described previously. The S0456-labeled Her2-targeted-4HBs is assessed by mass spectrometry and the optical properties of the conjugate (fluorescence and absorbance) is thoroughly characterized. Cell labeling is also assessed by fluorescence microscopy, using 4T1 breast cancer cells that have been engineered to overexpress the Her2/neu receptor and green fluorescent protein (GFP). 4T1 cells, which are Her2/neu-negative and GFP-positive, serve as a negative control. Competitive inhibition studies are also used as a second negative control. Analogous studies are performed with free S0456 and Her2-targeted affibodies that are labeled only at their c-terminus with S0456 (Her2-S0456).

### Evaluating the Accuracy of Tumor Margin Identification in a Murine Model of Breast Cancer

The ability of S0456-labeled Her2-targeted-4HBs to accurately identify tumor margins, in a syngeneic orthotopic 4T1 breast tumor model is evaluated. The accuracy of tumor demarcation is compared with free S0456 and Her2-S0456. Her2-positive, GFP-positive 4T1 cells are implanted orthotopically into the breast fat pad of mice (3 groups, 5 per group). Once tumors reach a size of —8mm, mice are injected (i.v) with S0456-labeled Her2-targete-4HBs, free S0456 or Her2-S0456. Fluorescent images are acquired 24 hours after injection. Animals are then sacrificed, and their mammary fat pads excised. The accuracy of determining the tumor margin is quantified by analyzing the fluorescent images of GFP and S0456, as previously described.

### Example 2

Breast Cancer Imaging: It is generally accepted that early detection of breast cancer can greatly improve the chances of patient survival. This is supported by clinical data, which has shown that mammography screening led to a 28-65% reduction in the rate of breast cancer mortality in the U.S. between 1975 and 2000. Despite the proven effectiveness of mammography, this method of screening has a sensitivity that can be as low as 75% and a specificity of 90-95%.

Recently, large-scale studies have demonstrated the value of magnetic resonance imaging (MRI) as an effective tool in the diagnosis of breast cancer. Specifically, gadolinium (Gd)-enhanced MRI can detect malignancies that are often missed by mammographies, with a sensitivity ranging from 88-95%. As a result, it is now recommended that women at high-risk for breast cancer receive yearly mammograms and supplemental MR breast scans. However, a major challenge for current MR breast scans is overcoming the low specificity, which is in the range of only 30-80%. Because MRI results in a significant number of biopsies of non-cancerous tissue, MR imaging is generally not recommended for women at average risk. It has been theorized that the development of actively targeted MR contrast agents with high relaxivity could significantly improve the specificity (and sensitivity) of tumor detection by providing insight into the molecular expression of breast lesions.

MRI contrast agents: Most Gd-based agents are small, non-targeted compounds that passively distribute into the intravascular and interstitial space with nonspecific biodistribution. As a result conditions such as intraglandular dysplasia, benign hyperplasia, post-biopsy hemorrhage, and therapeutic effects can all have a similar appearance on contrast-enhanced MR images. Specificity cannot be improved by directly functionalizing clinically-used Gd-based contrast agents with targeting ligands, because the sensitivity of individual Gd ions is too low. There are just an inadequate number of target receptors on the cell surface to allow a sufficient concentration of Gd to accumulate at the tumor site to generate MR contrast. To compensate for the low signal enhancement generated by individual Gd ions, most targeted gadolinium compounds have relied on the development of macromolecules and nanoplatforms that can carry a high payload of gadolinium and enhance the longitudinal relaxivities (R1) per gadolinium. Several systems have already been tested as platforms for Gd labeling, including dendrimers, polymers, emulsions, silica nanoparticles, and nanovesicles.

Tumor Penetration of Nanoparticles: A major obstacle faced by the use nanoparticles as MR contrast agents is their inability to penetrate tumors significantly beyond the vascular wall. The extracellular matrix (ECM) within tumors is composed of a dense collagen network embedded in a gel of glycosaminoglycans (GAGs), primarily hyaluronan, that can significantly impede the penetration of nanoparticles. The ECM creates both a physical barrier and a hydrodynamic barrier in the form of intratumoral pressure that prevents nanoparticles, due to their large size, from reaching tumor foci. This is considered to be one of the most significant barriers facing the entire field of nanomedicine.

As nanoparticles are made smaller, they are able to penetrate tumors more effectively, but the payload of Gd is significantly reduced. Therefore, a delicate balance must be maintained between utilizing a platform that is capable of carrying a sufficient Gd payload to generate MR contrast, but that is also small enough to penetrate tumor tissue with sufficient efficiency to reach tumor cells. Additional factors to be considered when designing new targeted MR contrast agents are the ability to synthesize the contrast agent with high homogeneity the ability to maintain precise control over their functionalization with tumor-specific targeting ligands.

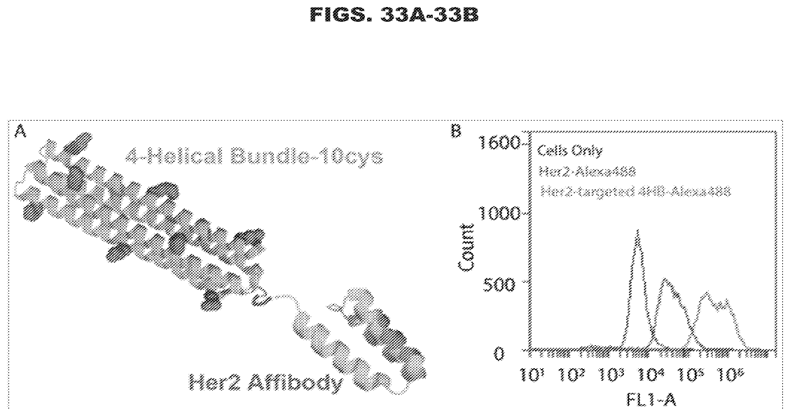

An MR contrast agent is created from a 4-helical bundle (4HB) protein (MW=23 kDa) that has been engineered to possess 6 to 10 cysteines at precisely defined locations (FIG. 33A). Each cysteine is labeled with a thiol-reactive dendron (generation 4) that can carry up to 16 Gd. Therefore, each 4HB will ultimately be labeled with up to 96 to 160 Gd. The Gd-labeled 4HBs can serve as a universal platform that can be conjugated or fused to any targeting ligand for contrast-enhanced molecular imaging. In this proposal, the 4HB will be fused to a Her2/neu-targeted affibody. Affibodies are small (6.5 kDa), robust molecules that exhibit remarkable specificity and affinity (pM range) for the HER2/neu receptor. Overexpression of the Her2/neu receptor has been associated with highly aggressive forms of breast cancer. The Her2-targeted 4HB fusions have been bacterially expressed and purified. Further, it has been confirmed that the cysteines on the 4HB can be efficiently and site-specifically labeled, using the thiol-reactive fluorescent dye, maleimide-Alexa488. The same conjugation strategy is used to label the Her2-targeted 4HBs with DOTA-terminated dendrons, which are subsequently loaded with Gd. To confirm the binding capability of the fluorescently-labeled Her2-targeted 4HBs, these fusion proteins were incubated with Her2-positive cells, and cell labeling was assessed via fluorescence microscopy and flow cytometry. Cells incubated with the Her2-targeted-4HBs exhibited a >5-times higher mean fluorescence than cells incubated with an equivalent dose of Her2-targeted affibodies labeled with a single Alexa488 at their c-terminus (FIG. 33B). This demonstrates the higher labeling capacity of the HB, compared with direct labeling of the targeting ligand. In this Example, the payload that each 4HB can carry is further expanded by labeling each cysteine with a dendron that can carry up to 16 Gd.

The Her2-targeted 4HB is labeled with Gd-labeled generation 4 dendrons, the Her2-targeted 4HB is expected to generate sufficient contrast to detect Her2-positive tumors in living subjects, via MRI. Moreover, it is expected that the 4HB will exhibit better tissue penetration and distribution, compared with fluorescent nanoparticles, due to the small size of the 4HB.

It is also expected that the Her2-targeted 4HB will also be small enough to be rapidly removed from circulation by renal filtration. Typically, this is desirable for imaging studies, because it allows for rapid imaging; however, if it is determined that a longer circulation time is needed to provide time to allow higher levels of tumor binding, the construct could easily be modified with PEG to extend circulation time. Since 4HB s are found throughout nature, including within many mammalian proteins (e.g. ferritin, human growth hormone, cytokines), these agents are expected to elicit little to no immunogenic response. Therefore, dendron-labeled 4HBs that have been loaded with Gd can serve as a universal, high contrast platform that can be coupled with any targeting agent for molecular imaging.
Prepare a Thiol-Reactive Generation 4 Dendron with DOTA Terminal Groups

A generation 4 dendron is synthesized with 16 terminal groups, each of which is labeled with the Gd-chelator, DOTA. A thiol-reactive maleimide group is placed at the focal point of the dendron.

### Prepare and Characterize Dendron-Labeled Her2-Targeted 4HBs

Her2-targeted 4HBs have been recombinantly expressed and purified as fusion proteins. The 4HB was engineered with 6 to 10 strategically placed cysteine residues. The 4HB is labeled with the thiol-reactive generation 4 dendron prepared above and subsequently loaded with Gd. Note: Neither the 4HB nor the affibody include cysteines in their native amino acid sequence. The dendron-labeled Her2-targeted-4HBs before and after loading with Gd (i.e., Her2-targeted 4HB-dendron(Gd)) is assessed by mass spectrometry and ICP-OES, respectively. Cell labeling is also assessed by MR imaging of cell pellets, using 4T1 breast cancer cells that have been engineered to overexpress the Her2/neu receptor. 4T1 cells, which are Her2/neu-negative, serves as a negative control. Competitive inhibition studies with an excess of unlabeled Her2-targeted affibody are also used as a second negative control. Analogous studies are performed with free Gd-DOTA, Her2-targeted affibodies are labeled at their c-terminus with a single Gd (Her2-Gd), and Her2-targeted-4HBs that have had each cysteine labeled with a single Gd (Her2-targeted 4HB-Gd).

Evaluate the Tumor Contrast Enhancement with Her2-Targeted 4HB-Dendron(Gd) in a Murine Model of Breast Cancer

The ability of Her2-targeted 4HB-dendron(Gd) to specifically detect Her2-positive tumors in a syngeneic orthotopic 4T1 breast tumor model is evaluated. The tumor-specific contrast enhancement is compared with free Gd-DOTA, Her2-Gd, and Her2-targeted 4HB-Gd. Her2-positive 4T1 cells are implanted orthotopically into the breast fat pad of mice (4 groups, 5 per group). Once tumors reach a size of ˜8 mm, mice are injected (i.v) with Her2-targeted 4HB-dendron(Gd), free Gd-DOTA, Her2-Gd, or Her2-targeted 4HB-Gd. MR images are acquired 24 hours after injection. Animals are then sacrificed, and their organs harvested for histological evaluation. Immunostaining is also performed to assess the intratumor distribution of the Her2-targeted 4HB-dendron(Gd).

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

What is claimed is:

1. A protein scaffold comprising: (i) a helical bundle having a plurality of chemical handles at defined locations and (ii) cargo, wherein the chemical handles have been labeled with the cargo.

2. The protein scaffold of claim 1, wherein the helical bundle is one that ranges from a 2-helical bundle to an 8-helical bundle.

3. The protein scaffold of claim 1, wherein the helical bundle is a 4-helical bundle (4HB).

4. The protein scaffold of claim 1, wherein the helical bundle comprises an amino acid sequence selected from an amino acid sequence set forth in SEQ ID NOs: 1-30.

5. The protein scaffold of claim 1, wherein the chemical handles are selected from the group consisting of a cysteine a lysine, an unnatural amino acid and combinations thereof.

6. The protein scaffold of claim 1, wherein the helical bundle has 3 to 50 chemical handles at the defined locations.

7. The protein scaffold of claim 1, wherein the cargo is selected from the group consisting of fluorescent dyes, haptens, contrast agents, chelated metals, therapeutic agents, sensitizers, small molecules, and combinations thereof.

8. The protein scaffold of claim 7, wherein the hapten is biotin.

9. The protein scaffold of claim 7, wherein the contrast agent is selected from gadolinium and/or a radionuclide.

10. The protein scaffold of claim 7, wherein the cargo comprises fluorescent dyes.

11. The protein scaffold of claim 10, wherein the chemical handles are spaced so as to limit quenching of the fluorescent dyes.

12. The protein scaffold of claim 10, wherein the fluorescent dyes emit photons.

13. The protein scaffold of claim 10, wherein the fluorescent dye is a photosensitizer that generates a reactive oxygen species.

14. The protein scaffold of claim 10, wherein the fluorescent dyes are near-infrared (NIR) fluorescent dyes.

15. The protein scaffold of claim 14, wherein the helical bundle is labeled with 3 to 50 NIR fluorescent dyes with little to no self-quenching.

16. The protein scaffold of claim 10, where the fluorescent dye is S0456.

17. The protein scaffold of claim 1, wherein the chemical handles comprise a plurality of a first chemical handle, and a single second chemical handle that is distinct from the first, wherein the first chemical handle is selected from a lysine, a cysteine, an unnatural amino acid or combination thereof; wherein the second chemical handle is selected from a lysine, a cysteine, an amine, a thiol, an unnatural amino acid, a click-chemistry group, a thiol-reactive moiety, or an amine-reactive moiety; and wherein the first chemical handle is labeled with the cargo; and wherein the second chemical handle allows for the attachment of said helical bundle to a protein, nucleic acid, small molecule, particle, or surface.

18. The protein scaffold of claim 1, wherein the protein scaffold comprises a plurality of helical bundles operably linked in tandem and labeled with the cargo.

19. A composition comprising a protein scaffold according to any one of claims 1 to 18 that has been operably linked to a moiety selected from a protein, a nucleic acid, a polymer, a lipid, a small molecule or a combination thereof.

20. The composition of claim 19, wherein the moiety is a targeting ligand.

21. The composition of claim 20, wherein the targeting ligand comprises an antibody.

22. The composition of claim 20, wherein the targeting ligand comprises an affibody.

23. The composition of claim 20, wherein the targeting ligand comprises a DARPin, a nanobody, a centyrin, or an antibody fragment.

24. The composition of claim 20, wherein the targeting ligand comprises an antibody-binding domain (AbBD).

25. The composition of claim 20, wherein the targeting ligand comprises a photoreactive antibody-binding domain (pAbBD).

26. The composition of claim 25, wherein the targeting ligand further comprises an antibody operably linked to the pAbBD.

27. A method for imaging tumor cells in a subject, comprising:
(a) administering to the subject a protein scaffold according to any one of claims 1 to 18 that is operably linked to a targeting ligand or a composition according to any one of claims 20 to 26, wherein the targeting ligand binds to a surface of the tumor cells; and
(b) visualizing the tumor cells by detecting the cargo of the protein scaffold.

28. The method of claim 27, wherein the subject has breast, ovarian, bladder, pancreatic, or stomach cancer.

29. The method of claim 28, wherein the subject has breast cancer.

30. The method of claim 27, wherein the tumor cells overexpress EGFR, and wherein the targeting ligand comprises an anti-EGFR antibody or affibody.

31. The method of claim 27, wherein the tumor cells overexpress HER2/neu, and wherein the targeting ligand is an anti-HER2/neu antibody or affibody.

32. The method of claim 27, wherein the tumor cells are visualized by magnetic resonance imaging (MRI).

33. A method for intraoperative optical image-guided surgery of a tumor in a subject, comprising:
(a) administering to the subject a protein scaffold according to any one of claims 1 to 18 that is operably linked to a targeting ligand or a composition according to any one of claims 20 to 26, wherein the targeting ligand binds to a surface of cancer cells of the tumor;
(b) visualizing the tumor and delineating intraoperative margins thereof during the surgery by detecting the cargo of the protein scaffold; and
(c) resecting the tumor at or near the delineated intraoperative margins thereof.

34. The method of claim 33, wherein the subject has breast, ovarian, bladder, pancreatic, or stomach cancer.

35. The method of claim 34, wherein the subject has breast cancer.

36. The method of claim 33, wherein the cancer cells overexpress EGFR, and wherein the targeting ligand comprises an anti-EGFR antibody or affibody.

37. The method of claim 33, wherein the cancer cells overexpress HER2/neu, and wherein the targeting ligand is an anti-HER2/neu antibody or affibody.
